(19) United States
(12) Patent Application Publication
(10) Pub. No.: US 2024/0316008 A1
(43) Pub. Date: Sep. 26, 2024

(54) TREATMENT OF SYSTEMIC IMMUNE ACTIVATION SYNDROMES

(71) Applicant: TRANQUIS THERAPEUTICS, INC., Redwood City, CA (US)

(72) Inventor: Sanjay Kumar KAKKAR, London (GB)

(21) Appl. No.: 18/003,153

(22) PCT Filed: Jun. 21, 2021
(86) PCT No.: PCT/US2021/038313

§ 371 Date: Dec. 22, 2022

Related U.S. Application Data
(60) Provisional application No. 63/042,152, filed on Jun. 22, 2020.

(51) Int. Cl. A61K 31/4184; A61P 37/06

(52) U.S. Cl. CPC A61K 31/4184; A61P 37/06

(57) ABSTRACT

The disclosure provides methods of treating a patient who has, or who is at risk for developing, systemic immune activation, including cytokine release syndrome (CRS) and sepsis. The method comprises administering a therapeutically effective amount of a compound of Formula I the patient.

### CROSS REFERENCE TO RELATED APPLICATIONS

This application claims priority to U.S. Provisional Application No. 63/042,152, filed Jun. 22, 2020, which is incorporated herein by reference in its entirety.

### BACKGROUND

Cytokine release syndrome (CRS) is a systemic immune activation syndrome with high morbidity and mortality. CRS occurs when macrophages, monocytes, dendritic cells, T cells and other cells of the innate and adaptive immune system are activated and release inflammatory cytokines. IL-6, IL-10, and interferon (IFN)-γ are among the pro-inflammatory cytokines that are consistently found to be elevated in serum of patients with CRS. CRS can present with a variety of symptoms ranging from mild flu-like symptoms to severe life-threatening manifestations of the uncontrolled inflammatory response. CRS can be triggered by infection with a virus, such as an influenza virus or a coronavirus, and by therapeutic interventions that activate the immune system, such as CAR-T therapy.

Sepsis occurs when an infection escapes local tissue containment and induces dysregulated physiologic responses that result in organ dysfunction. A subset of patients with sepsis progress to septic shock, which is defined by profound circulatory, cellular, and metabolic abnormalities, and which is associated with increased mortality. During sepsis, systemic activation of the innate immune system by pathogen-associated molecular patterns and damage-associated molecular patterns results in a severe and persistent inflammatory response characterized by an excessive release of inflammatory cytokines such as IL-1, TNF, and IL-17.

There is a need for new methods for treating or preventing systemic immune activation syndromes, including CRS and sepsis.

### SUMMARY

TQS-168 (2-(4-tert-butylphenyl)-1H-benzimidazole), previously known as ZLN-005, is known to be an activator of Ppargc1α (PGC-1α) expression (Zhang et al., Diabetes 62:1297-1307 (2013)). TQS-168 has previously been shown to suppress myeloid-mediated inflammation and reduce disease severity in murine models of neurodegenerative diseases in which neuroinflammation contributes to the underlying pathophysiology, including Parkinson's disease, Alzheimer's disease, 37543/46077/FW/16492279.1 and amyotrophic lateral sclerosis (ALS) (U.S. Pat. Nos. 10,272,070; 10,583,125; and 10,653,669, the disclosures of which are incorporated herein by reference in their entireties).

PGC-1α is a transcriptional coactivator; induction of PGC-1α expression is therefore upstream of, and only indirectly related to, the observed in vivo anti-inflammatory activities of TQS-168.

We have now demonstrated that TQS-168 and structurally related analogs are capable of inhibiting secretion of inflammatory cytokines from LPS-stimulated murine microglial cells and from human PBMCs at concentrations that increase PGC-1α expression. LPS is a canonical trigger for inducing sepsis. TQS-168 and analogs inhibit secretion of both TNF-α and IL-6. IL-6 is a known mediator of CRS, and IL-6 inhibitors are currently being trialed for treatment and prevention of CRS in patients infected with SARS-CoV-2.

Using the Eurofins BioMap screening platform, we have further demonstrated that TQS-168 is anti-proliferative to endothelial, B cells, T cells, coronary artery smooth muscle cell fibroblasts. Pulmonary and other end-organ fibrosis is now believed to contribute to the morbidity of infections with SARS-CoV-2 (COVID-19). Spagnolo et al., The Lancet: Respiratory Medicine (doi.org/10.1016/S2213-2600(20)30222-8) (2020).

Accordingly, in a first aspect, methods are provided of treating a patient who has, or who is at risk for developing, systemic immune activation.

In some embodiments, the method comprises administering to the patient an effective amount of a compound of Formula I:

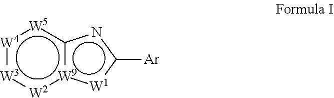

or a salt, hydrate, deuterated analog, or fluorinated analog thereof,
wherein:
Ar is

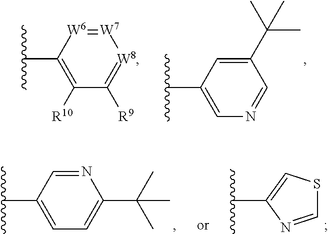

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

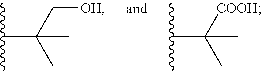

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

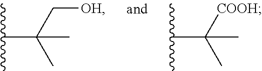

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxoalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, systemic immune activation does not comprise clinically meaningfully neuroinflammation.

In some embodiments, systemic immune activation does not comprise clinically significant neuroinflammation.

In some embodiments, the compound of Formula I is selected from:

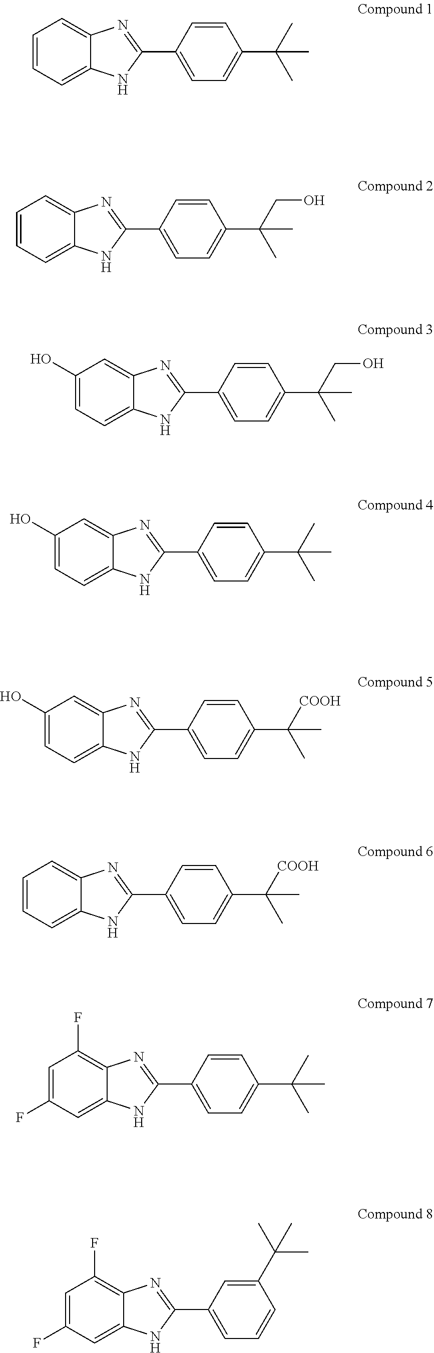

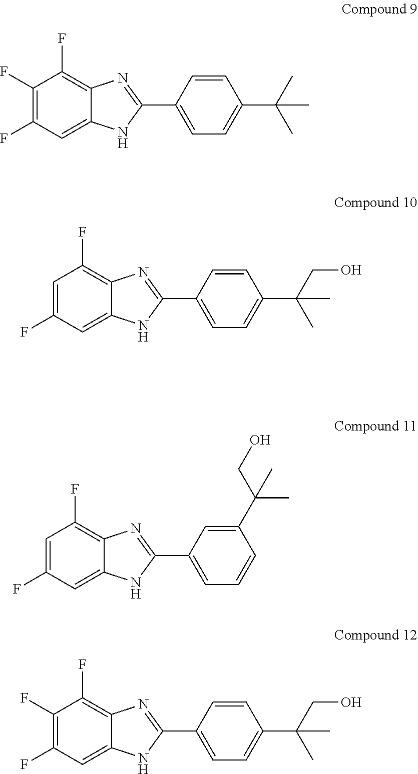 [SMILES: CC(C)(C)c1ccc(-c2nc3c(F)c(F)c(F)cc3[nH]2)cc1 ; CC(C)(CO)c1ccc(-c2nc3c(F)cc(F)cc3[nH]2)cc1 ; CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]

or a salt, hydrate, deuterated analog, or fluorinated analog thereof.

In some embodiments, the compound of Formula I is compound 1, according to the formula:

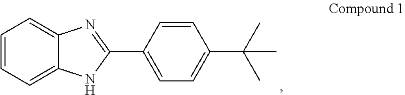 [SMILES: CC(C)(C)c1ccc(-c2nc3ccccc3[nH]2)cc1]

or a salt, hydrate, deuterated analog, or fluorinated analog thereof.

In some embodiments, the compound of Formula I is compound 8, according to the formula:

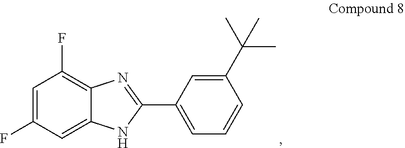 [SMILES: CC(C)(C)c1cccc(-c2nc3c(F)cc(F)cc3[nH]2)c1]

or a salt, hydrate, deuterated analog, or fluorinated analog thereof.

In some embodiments, the compound of Formula I is compound 7, according to the formula:

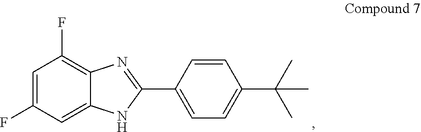 [SMILES: CC(C)(C)c1ccc(-c2nc3c(F)cc(F)cc3[nH]2)cc1]

or a salt, hydrate, deuterated analog, or fluorinated analog thereof.

In some embodiments, the compound of Formula I is compound 9, according to the formula:

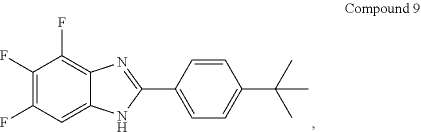 [SMILES: CC(C)(C)c1ccc(-c2nc3c(F)c(F)c(F)cc3[nH]2)cc1]

or a salt, hydrate, deuterated analog, or fluorinated analog thereof.

In some embodiments, the compound of Formula I is compound 2, according to the formula:

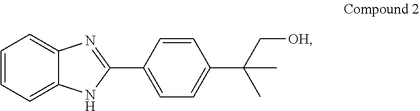 [SMILES: CC(C)(CO)c1ccc(-c2nc3ccccc3[nH]2)cc1]

or a salt, hydrate, deuterated analog, or fluorinated analog thereof.

In some embodiments, the patient has cytokine release syndrome (CRS).

In some embodiments, the patient has acute lung inflammation (ALI).

In some embodiments, the patient has acute respiratory distress syndrome (ARDS).

In some embodiments, the patient has ALI with concomitant pneumonia or ARDS with concomitant pneumonia.

In some embodiments, the patient has acute renal injury.

In some embodiments, the patient has ischemia-reperfusion injury (IRI) or reoxygenation injury.

In some embodiments, the IRI is associated with coronary ischemia, brain ischemia, renal ischemia, or intestinal ischemia, in the patient.

In some embodiments, the patient has sepsis.

In some embodiments, the patient has a stroke.

In some embodiments, the patient has a confirmed or suspected viral infection.

In some embodiments, the infection is by a virus selected from the group consisting of coronavirus, influenza virus, rhinovirus, respiratory syncytial virus, metapneumovirus, adenovirus, and boca virus.

In some embodiments, the virus is a coronavirus selected from the group consisting of coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19).

In some embodiments, the coronavirus is SARS-CoV-2 (COVID-19).

In some embodiments, the virus is an influenza virus selected from the group consisting of parainfluenza virus 1, parainfluenza virus 2, influenza A virus, and influenza B virus.

In some embodiments, the patient has confirmed or suspected hypercoagulability.

In some embodiments, the patient is not hospitalized.

In some embodiments, the patient is hospitalized.

In some embodiments, the patient is not on a ventilator.

In some embodiments, the compound of Formula I is administered intravenously or enterically.

In some embodiments, the compound of Formula I is administered by mouth (p.o.).

In some embodiments, the compound of Formula I is administered via an enteral feeding tube.

In some embodiments, the enteral feeding tube is a nasogastric tube.

In some embodiments, the compound of Formula I is mixed with an enteral feeding formula.

In some embodiments, the effective amount of the compound of Formula I is between 0.5 mg/kg and 85 mg/kg per day by mouth.

In some embodiments, the dose is 2 mg/kg.

In some embodiments, the dose is administered as a single daily dose.

In some embodiments, the dose is administered as a plurality of equally or unequally divided sub-doses.

In some embodiments, the dose is administered as a plurality of unequally divided sub-doses.

In some embodiments, the patient has a body temperature greater than 37.5° C. prior to first administration of the compound of Formula I, or a salt, hydrate, deuterated analog, or fluorinated analog thereof.

In some embodiments, the body temperature of the patient is measured at one or more sites selected from the group consisting of an oral cavity, a rectal cavity, axilla area, and tympanic membrane.

In some embodiments, the patient has a pre-treatment C-reactive protein (CRP) level greater than 2 mg/L.

In some embodiments, the patient has a pre-treatment CRP level greater than 5 mg/L, 10 mg/L, 15 mg/L, 20 mg/L, 25 mg/L, 30 mg/L, 35 mg/L, or 40 mg/L.

In some embodiments, the patient has a pre-treatment serum IL-6 level of at least 2 pg/ml.

In some embodiments, the patient has a pre-treatment serum IL-6 level of at least 2.5 pg/ml, 3 pg/ml, 4 pg/ml, 5 pg/ml, 10 pg/ml, 20 pg/ml, 30 pg/ml, 40 pg/ml, 50 pg/ml, 60 pg/ml, 70 pg/ml, 80 pg/ml, 90 pg/ml, 100 pg/ml, 150 pg/ml or 200 pg/ml.

In some embodiments, the patient has a pre-treatment neutrophil-to-lymphocyte ratio (NLR) greater than 2.0.

In some embodiments, the patient has a pre-treatment NLR greater than 2.8, 2.9, 3.0, 3.1, 3.2, 3.3, 3.4, 3.5, 3.6, 3.7, 3.8, 3.9, or 4.0.

In some embodiments, the patient has a pre-treatment respiration rate on ambient air of fewer than 12 breaths or more than 25 breaths per minute.

In some embodiments, the patient has a pre-treatment oxygen saturation level on ambient air of no more than 95%, 94%, 93%, 92%, 91%, 90%, 89%, 88%, 87%, 86%, 85%, 84%, 83%, 82%, 81%, 80%, or 75%.

In some embodiments, the patient has a pre-treatment D-Dimer level that is elevated above baseline.

In some embodiments, the patient has a pre-treatment sepsis-induced coagulopathy (SIC) total score of 4 or more with total score of prothrombin time and coagulation exceeding 2.

In some embodiments, the method reduces the body temperature of the patient below pre-treatment levels.

In some embodiments, the method reduces the patient's serum CRP levels below pre-treatment levels.

In some embodiments, the post-treatment CRP level is no more than 45 mg/L, 40 mg/L, 35 mg/L, 30 mg/L, 25 mg/L, 20 mg/L, 15 mg/L, 10 mg/L, 5 mg/L, or 1 mg/L.

In some embodiments, the method reduces the CRP level by at least 10%, 20%, 30%, 40%, 50%, 60%, 70%, 80%, or 90% as compared to pre-treatment levels.

In some embodiments, the method reduces, in the patient, one or more pro-inflammatory cytokine serum levels below pre-treatment levels, wherein the one or more pro-inflammatory cytokines is selected from the group consisting of: IL-6, TNFα; IL-17A; IL-17F, and IL-2.

In some embodiments, the method reduces the patient's serum IL-6 levels below pre-treatment levels.

In some embodiments, the serum IL-6 level is decreased by at least 10% as compared to pre-treatment levels.

In some embodiments, the serum IL-6 level is decreased by at least 20%, 30%, 40%, 50%, 60%, 70%, 80%, or 90% as compared to pre-treatment levels.

In some embodiments, the serum TNFα level is decreased by at least 10% as compared to pre-treatment levels.

In some embodiments, the serum TNFα level is decreased by at least 20%, 30%, 40%, 50%, 60%, 70%, 80%, or 90% as compared to pre-treatment levels.

In some embodiments, the patient has a reduced serum level of immunoglobulin G (IgG) as compared to pre-treatment levels.

In some embodiments, the patient has a reduced serum level of immunoglobulin G (IgG) by at least 10% as compared to pre-treatment levels.

In some embodiments, the patient has a post-treatment NLR less than 3.18.

In some embodiments, the administration of the compound of Formula I improves the NLR by at least 10%, 20%, 30%, 40%, 50%, 60%, 70%, 80%, or 90% as compared to pre-treatment levels.

In some embodiments, the method improves the respiration rate of the patient.

In some embodiments, the patient has a post-treatment respiration rate between 12 to 20 breaths per minute.

In some embodiments, the method improves the oxygen saturation level of the patient on ambient air by 1%, 2%, 3%, 4%, 5%, 6%, 7%, 8%, 9%, 10%, 11%, 12%, 13%, 14%, 15%, 16%, 17%, 18%, 19%, 20%, 25%, or 30% as compared to pre-treatment levels.

In some embodiments, the method reduces the patient's need for supplemental oxygen.

In some embodiments, the patient is older than 50, 51, 52, 53, 54, 55, 56, 57, 58, or 59 years of age.

In some embodiments, the patient is older than 60 years of age.

In some embodiments, the patient is younger than 60, 59, 58, 57, 56, 55, 54, 53, 52, 51, or 50 years of age.

In some embodiments, the administration of the compound of Formula I, or a salt, hydrate, deuterated analog, or fluorinated analog thereof increases the expression level of Ppargc1α (PCG-1 α) in the lungs as compared to pre-treatment levels.

In some embodiments, the method further comprises administering an effective amount of at least one second therapeutic agent selected from the group consisting of an antiviral agent, an antibacterial agent, an IL-6 antagonist, an angiotensin receptor blocker (ARB), granulocyte/macrophage colony stimulating factor (GM-CSF) antagonist, hydroxychloroquine, chloroquine, and COVID-19 immune serum or plasma.

In some embodiments, the at least one second therapeutic agent is an antiviral agent.

In some embodiments, the antiviral agent is favipiravir.

In some embodiments, the antiviral agent is remdesivir.

In some embodiments, the at least one second therapeutic agent is an antibacterial agent selected from the group consisting of azithromycin, tobramycin, aztreonam, ciprofloxacin, meropenem, cefepime, cetadizine, imipenem, piperacillin-tazobactam, amikacin, gentamicin, and levofloxacin.

In some embodiments, the at least one second therapeutic agent is an IL-6 antagonist selected from the group consisting of an anti-IL-6 antibody or an antigen binding fragment thereof, an anti-IL-6 receptor antibody or an antigen binding fragment thereof, and a JAK/STAT inhibitor.

In some embodiments, the at least one second therapeutic agent is a GM-CSF antagonist.

In some embodiments, the GM-CSF antagonist is gemsilumab.

In a second aspect, the present disclosure provides methods, comprising:
administering an effective amount of compound 1, according to the formula

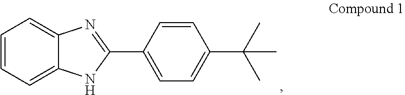 [SMILES: CC(C)(C)c1ccc(-c2nc3ccccc3[nH]2)cc1]

or a salt, hydrate, deuterated analog, or fluorinated analog thereof to a patient who has, or is at risk for developing, systemic immune activation, wherein the patient does not have a neurodegenerative disorder.

In some embodiments, the systemic immune activation does not comprise clinically significant neuroinflammation.

In some embodiments, the patient has or is at risk for peripheral inflammation.

In some embodiments, the patient has confirmed or suspected hypercoagulability.

In some embodiments, the patient is hospitalized.

In some embodiments, the effective amount of compound 1, or a salt, hydrate, deuterated analog, fluorinated analog thereof is administered intravenously or enterically.

In some embodiments, compound 1, or a salt, hydrate, deuterated analog, fluorinated analog thereof is administered by mouth (p.o.).

In some embodiments, compound 1, or a salt, hydrate, deuterated analog, fluorinated analog thereof is administered via an enteral feeding tube.

In some embodiments, the effective amount of compound 1, or a salt, hydrate, deuterated analog, fluorinated analog thereof is between 0.5 mg/kg to 85 mg/kg per day.

In some embodiments, the dose is administered enterically.

In some embodiments, the dose is administered as a single daily dose.

In some embodiments, the dose is administered as a plurality of equally or unequally divided sub-doses.

In some embodiments, the patient has a body temperature greater than 37.5° C. prior to first administration of compound 1, or a salt, hydrate, deuterated analog, or fluorinated analog thereof.

In some embodiments, the body temperature of the patient is measured at one or more sites selected from the group consisting of an oral cavity, a rectal cavity, axilla area, and tympanic membrane.

In some embodiments, the patient has a pre-treatment C-reactive protein (CRP) level greater than 2 mg/L.

In some embodiments, the patient has a pre-treatment serum IL-6 level of at least 2 pg/ml.

In some embodiments, the patient has a pre-treatment neutrophil-to-lymphocyte ratio (NLR) greater than 2.0.

In some embodiments, the patient has a pre-treatment respiration rate on ambient air of fewer than 12 breaths or more than 25 breaths per minute.

In some embodiments, the patient has a pre-treatment oxygen saturation level on ambient air of no more than 95%, 94%, 93%, 92%, 91%, 90%, 89%, 88%, 87%, 86%, 85%, 84%, 83%, 82%, 81%, 80%, or 75%.

In some embodiments, the patient has a pre-treatment D-Dimer level that is elevated above baseline.

In some embodiments, the patient has a pre-treatment sepsis-induced coagulopathy (SIC) total score of 4 or more with total score of prothrombin time and coagulation exceeding 2.

In some embodiments, the method reduces the patient's serum CRP levels below pre-treatment levels.

In some embodiments, the post-treatment CRP level is no more than 45 mg/L, 40 mg/L, 35 mg/L, 30 mg/L, 25 mg/L, 20 mg/L, 15 mg/L, 10 mg/L, 5 mg/L, or 1 mg/L.

In some embodiments, the method reduces the CRP level by at least 10%, 20%, 30%, 40%, 50%, 60%, 70%, 80%, or 90% as compared to pre-treatment levels.

In some embodiments, the method reduces, in the patient, one or more pro-inflammatory cytokine serum levels below pre-treatment levels, wherein the one or more pro-inflammatory cytokines is selected from the group consisting of: IL-6, TNFα; IL-17A; IL-17F, and IL-2.

In some embodiments, the serum TNFα level is decreased by at least 10% as compared to pre-treatment levels.

In some embodiments, the serum TNFα level is decreased by at least 20%, 30%, 40%, 50%, 60%, 70%, 80%, or 90% as compared to pre-treatment levels.

In some embodiments, the method reduces the patient's serum IL-6 levels below pre-treatment levels.

In some embodiments, the serum IL-6 level is decreased by at least 10% as compared to pre-treatment levels.

In some embodiments, the patient has a reduced serum level of immunoglobulin G (IgG) as compared to pre-treatment levels.

In some embodiments, the patient has a reduced serum level of immunoglobulin G (IgG) by at least 10% as compared to pre-treatment levels.

In some embodiments, the patient has a post-treatment NLR less than 3.18.

In some embodiments, the administration of compound 1, or a salt, hydrate, deuterated analog, or fluorinated analog thereof improves the NLR by at least 10%, 20%, 30%, 40%, 50%, 60%, 70%, 80%, or 90% as compared to pre-treatment levels.

In some embodiments, the method improves the respiration rate of the patient.

In some embodiments, the patient has a post-treatment respiration rate between 12 to 20 breaths per minute.

In some embodiments, the method improves the oxygen saturation level of the patient on ambient air by at least 1%, 2%, 3%, 4%, 5%, 6%, 7%, 8%, 9%, 10%, 11%, 12%, 13%, 14%, 15%, 16%, 17%, 18%, 19%, 20%, 25%, or 30% as compared to pre-treatment levels.

In some embodiments, the method reduces the patient's need for supplemental oxygen.

In some embodiments, the patient is older than 50, 51, 52, 53, 54, 55, 56, 57, 58, or 59 years of age.

In some embodiments, the patient is older than 60 years of age.

In some embodiments, the patient is younger than 60, 59, 58, 57, 56, 55, 54, 53, 52, 51, or 50 years of age.

In some embodiments, the administration of compound 1, or a salt, hydrate, deuterated analog, or fluorinated analog thereof increases the expression level of Ppargc1α (PCG-1 α) in the lungs as compared to pre-treatment levels.

In some embodiments, the method reduces the body temperature of the patient below pre-treatment levels.

In some embodiments, the method further comprises administering an effective amount of at least one second therapeutic agent selected from the group consisting of an antiviral agent, an antibacterial agent, an IL-6 antagonist, an angiotensin receptor blocker (ARB), granulocyte/macrophage colony stimulating factor (GM-CSF) antagonist, hydroxychloroquine, chloroquine, and COVID-19 immune serum or plasma.

In some embodiments, the at least one second therapeutic agent is an antiviral agent.

In some embodiments, the antiviral agent is favipiravir.

In some embodiments, the antiviral agent is remdesivir.

In some embodiments, the at least one second therapeutic agent is an antibacterial agent selected from the group consisting of azithromycin, tobramycin, aztreonam, ciprofloxacin, meropenem, cefepime, cetadizine, imipenem, piperacillin-tazobactam, amikacin, gentamicin, and levofloxacin.

In some embodiments, the antibacterial agent is azithromycin.

In some embodiments, the at least one second therapeutic agent is an IL-6 antagonist selected from the group consisting of an anti-IL-6 antibody or an antigen binding fragment thereof, an anti-IL-6 receptor antibody or an antigen binding fragment thereof, and a JAK/STAT inhibitor.

In some embodiments, the at least one second therapeutic agent is an ARB.

In some embodiments, the ARB is losartan

In some embodiments, the ARB is valsartan

In some embodiments, the at least one second therapeutic agent is a GM-CSF antagonist.

In some embodiments, the GM-CSF antagonist is gemsilumab.

In a third aspect, methods are provided of treating a patient who has, or who is at risk for developing, sepsis. In some embodiments, the method comprises administering to the patient an effective amount of a compound of Formula I:

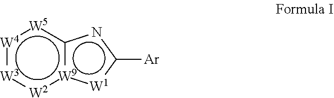

or a salt, hydrate, deuterated analog, or fluorinated analog thereof,
wherein:
Ar is

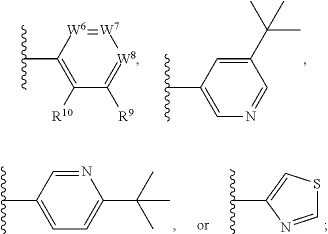

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

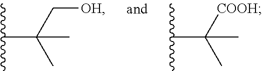

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

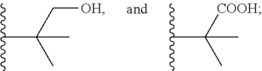

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

### BRIEF DESCRIPTION OF THE DRAWINGS

These and other features, aspects, and advantages of the present invention will become better understood with regard to the following description, and accompanying drawings, where:

FIG. 1 is a Western blot showing induction of PGC-1α protein expression in the BV2 murine microglial cell line after contact with TQS-168 at 20 μM in vitro.

FIG. 2 is a Western blot showing the dose response of the increase in PGC-1α protein expression in BV2 cells contacted with TQS-168 in vitro.

FIG. 3 is a bar graph quantifying expression levels of Ppargc1α mRNA levels in BV2 cells contacted with TQS-168 in vitro.

FIGS. 4a-4f show the in vitro secretion of several cytokines by BV2 murine microglial cells after LPS activation, as measured using a cytokine bead array (CBA) FACs assay. TQS-168 is capable of inhibiting TNFα and IL-6 cytokine release by LPS-stimulated BV2 cells at various concentrations.

FIGS. 5a-5b show inhibition of TNFα production by LPS-stimulated BV2 cells in vitro by 5 μM (FIG. 5a) and 20 μM (FIG. 5b) TQS-168 treatments, respectively.

FIGS. 6a-6d show the dose response of TQS-168-mediated decrease of pro-inflammatory cytokine TNFα release from LPS-stimulated BV2 cells at 24 hours using ELISA. FIG. 6a and FIG. 6c show the absolute (FIG. 6a) and relative (FIG. 6c) inhibition of TNFα secretion by BV2 cells stimulated with 0.3 ng/ml of LPS. FIG. 6b and FIG. 6d show the absolute (FIG. 6b) and relative (FIG. 6d) inhibition of TNFα secretion by BV2 cells stimulated with 1 ng/ml of LPS.

FIG. 7 shows inhibition of LPS-induced TNFα secretion in human PBMC cells treated in vitro with TQS-168 at various concentrations.

FIG. 8 shows concentration-response curves for inhibition of TNFα secretion from LPS-stimulated BV2 cells by TQS-168 and various analogs, with FIG. 8A graphing inhibition of TNFα secretion and FIG. 8B graphing inhibition of IL-6 secretion.

FIG. 9 compares the ability of TQS-168 and TQS-237 to inhibit secretion of cytokine by BV2 cells stimulated with LPS, with FIG. 9A comparing inhibition of TNFα secretion and FIG. 9B comparing inhibition of IL-6 secretion.

FIG. 10 compares the ability of TQS-168 and TQS-240 to inhibit secretion of cytokine by BV2 cells stimulated with LPS, with FIG. 10A comparing inhibition of TNFα secretion and FIG. 10B comparing inhibition of IL-6 secretion.

FIG. 11 compares the ability of TQS-168 and TQS-235 to inhibit secretion of cytokine by BV2 cells stimulated with LPS, with FIG. 11A comparing inhibition of TNFα secretion and FIG. 11B comparing inhibition of IL-6 secretion.

FIG. 12 schematizes the systems surveyed in the Eurofins BioMAP Diversity PLUS® Panel.

FIG. 13 shows the BioMAP profile of TQS-168 with top concentration of 20 μM.

FIG. 14 shows the BioMAP profile of TQS-168 with top concentration of 6.7 μM.

FIG. 15 overlays BioMAP profiles of TQS-168 at 20 μM with the top similarity match, cerivastatin at 1.1 μM.

FIG. 16 overlays BioMAP profiles of TQS-168 at 6.7 μM, the highest nontoxic concentration tested, with the top similarity match, deferoxamine mesylate at 13 μM.

FIG. 17 overlays BioMAP profiles of TQS-168 at 6.7 μM with metformin at 5 μM.

FIG. 18 illustrates the phase 1 metabolites from liver metabolism of TQS-168 following oral administration.

FIG. 19A shows absolute inhibition of LPS-stimulated IL-6 secretion by TQS-168 and metabolite TQS-621 from previously frozen PBMCs obtained from a first healthy human volunteer donor. FIG. 19B shows the relative inhibition of IL-6 inhibition expressed as percentage activity.

FIG. 20 shows ex-vivo inhibition by TQS-168 and metabolite TQS-621 of the spontaneous release of IL-6 from PBMCs from an ALS patient.

### DETAILED DESCRIPTION

### 1. Definitions

Unless defined otherwise, all technical and scientific terms used herein have the meaning commonly understood by one of ordinary skill in the art to which the invention pertains.

A “therapeutically effective amount” of a composition is an amount sufficient to achieve a desired therapeutic effect, and therefore does not require cure or complete remission.

As used herein “preventing” a disease refers to inhibiting the full development of a disease.

As used herein, “interleukin 6 (IL-6)” or “IL-6 polypeptide” refers to a human polypeptide or fragment thereof having at least about 85% or greater amino acid identity to the amino acid sequence provided at NCBI Accession No. NP_000591 and having IL-6 biological activity. IL-6 is a pleotropic cytokine with multiple biologic functions. Exemplary IL-6 biological activities include immunostimulatory and pro-inflammatory activities.

As used herein, the term “IL-6 mediated inflammation” or “IL-6 mediated inflammatory disorder” refers to inflammation or inflammation related disorder in which IL-6 is known or suspected to contribute to the etiology or symptoms of the inflammation.
Unless otherwise specified, “IL-6 antagonist” is used synonymously with “IL-6 inhibitor” and refers to an agent that is capable of decreasing the biological activity of IL-6. IL-6 antagonists include agents that decrease the level of IL-6 polypeptide in serum, including agents that decrease the expression of an IL-6 polypeptide or nucleic acid; agents that decrease the ability of IL-6 to bind to the IL-6R; agents that decrease the expression of the IL-6R; and agents that decrease signal transduction by the IL-6R receptor when bound by IL-6. In preferred embodiments, the IL-6 antagonist decreases IL-6 biological activity by at least about 10%, 20%, 30%, 50%, 70%, 80%, 90%, 95%, or even 100%. As further described below, IL-6 antagonists include IL-6 binding polypeptides, such as anti-IL-6 antibodies and antigen binding fragments or derivatives thereof; IL-6R binding polypeptides, such as anti-IL-6R antibodies and antigen binding fragments or derivatives thereof; and synthetic chemical molecules, such as JAK1 and JAK3 inhibitors.

The term “IL-6 antibody” or “anti-IL-6 antibody” refers to an antibody that specifically binds IL-6 ligand. Anti-IL-6 antibodies include monoclonal and polyclonal antibodies that are specific for IL-6 ligand, and antigen-binding fragments or derivatives thereof. IL-6 antibodies are described in greater detail below.

The term “C-reactive protein” or “CRP” refers to a polypeptide or fragment thereof having at least about 85% or greater amino acid identity to the amino acid sequence provided at NCBI Accession No. NP_000558 and having complement activating activity. CRP levels increase in response to inflammation, and can be measured with an hsCRP (high-sensitivity C-reactive protein) test. An exemplary CRP sequence is provided below:

| (SEQ ID NO: 5) | |
|---|---|
| 1 | MEKLLCFLVL TSLSHAFGQT DMSRKAFVFP KESDTSYVSL KAPLTKPLKA FTVCLHFYTE |
| 61 | LSSTRGYSIF SYATKRQDNE ILIFWSKDIG YSFTVGGSEI LFEVPEVTVA PVHICTSWES |
| 121 | ASGIVEFWVD GKPRVRKSLK KGYTVGAEAS IILGQEQDSF GGNFEGSQSL VGDIGNVNMW |
| 181 | DFVLSPDEIN TIYLGGPFSP NVLNWRALKY EVQGEVFTKP QLWP |

The term “agent” refers to any compound or composition suitable to be administered in therapy, and explicitly includes chemical compounds; proteins, including antibodies or antigen-binding fragments thereof; peptides; and nucleic acid molecules.

The term “subject” refers to a human or non-human mammal, including, but not limited to, bovine, equine, canine, ovine, feline, and rodent, including murine and rattus, subjects. A “patient” is a human subject in need of treatment.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of a compound of Formula I according the methods described herein. Pre-treatment does not exclude, and often includes, the prior administration of treatments other than a compound of Formula I.

As used herein, “post-treatment” means after the administration of an TQS-168 compound according the methods described herein. Post-treatment includes after any administration of an TQS-168 compound at any dosage described herein. Post-treatment also includes after the treatment phase of TQS-168 compound.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

The term “biological sample” refers to any tissue, cell, fluid, or other material derived from an organism (e.g., human subject). In certain embodiments, the biological sample is serum or blood.

### 2. Methods of Treating or Preventing Systemic Immune Activation

In a first aspect, methods of treating a patient who has, or who is at risk for developing, systemic immune activation are presented. The methods comprise administering to the patient an effective amount of a compound of Formula I:

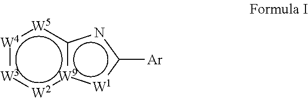

or a salt, hydrate, deuterated analog, or fluorinated analog thereof,
wherein:
Ar is

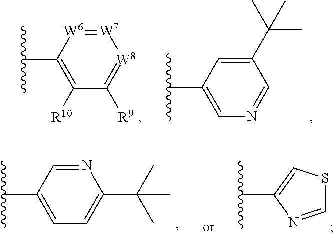

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

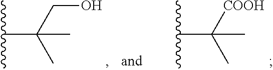

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

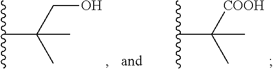

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, the compound of Formula I is selected from:

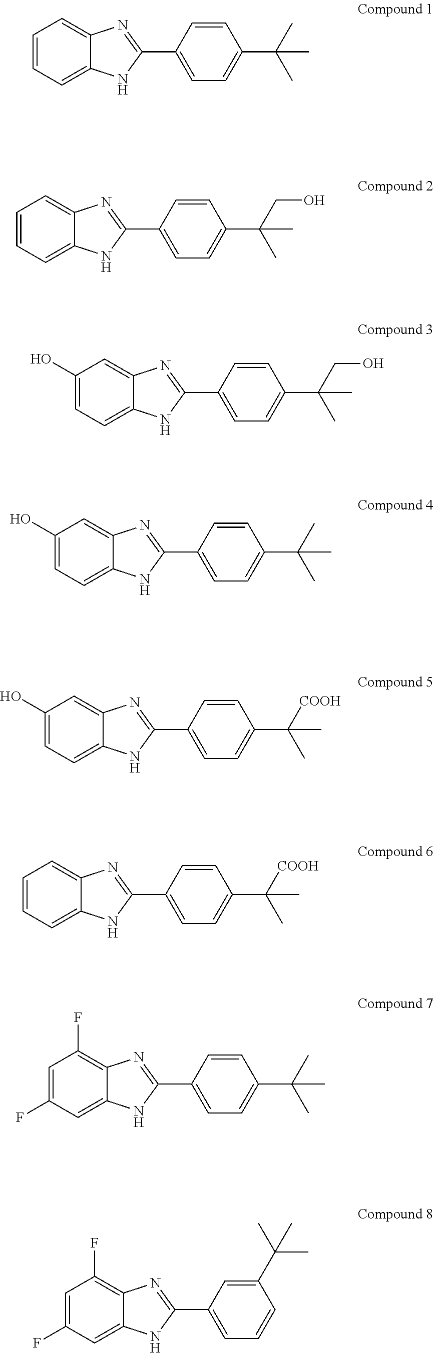

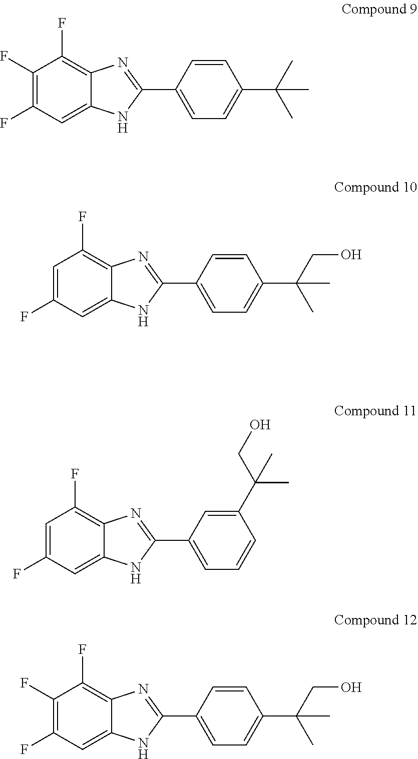 [SMILES: CC(C)(C)c1ccc(-c2nc3c(F)c(F)c(F)cc3[nH]2)cc1 ; CC(C)(CO)c1ccc(-c2nc3c(F)cc(F)cc3[nH]2)cc1 ; CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]

or a salt, hydrate, deuterated analog, or fluorinated analog thereof.

In particular embodiments, the compound of Formula I is compound 1, according to the formula:

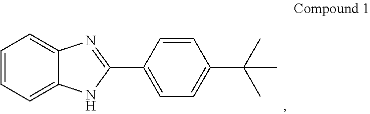 [SMILES: CC(C)(C)c1ccc(-c2nc3ccccc3[nH]2)cc1]

or a salt, hydrate, deuterated analog, or fluorinated analog thereof.

In particular embodiments, the compound of Formula I is compound 8, according to the formula:

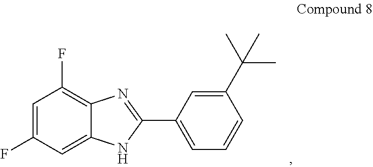 [SMILES: CC(C)(C)c1cccc(-c2nc3c(F)cc(F)cc3[nH]2)c1]

or a salt, hydrate, deuterated analog, or fluorinated analog thereof.

In particular embodiments, the compound of Formula I is 7, according to the formula:

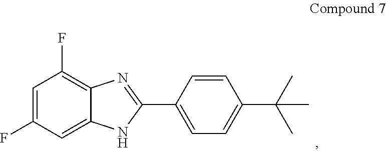 [SMILES: CC(C)(C)c1ccc(-c2nc3c(F)cc(F)cc3[nH]2)cc1]

or a salt, hydrate, deuterated analog, or fluorinated analog thereof.

In particular embodiments, the compound of Formula I is compound 9, according to the formula:

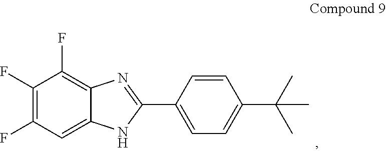 [SMILES: CC(C)(C)c1ccc(-c2nc3c(F)c(F)c(F)cc3[nH]2)cc1]

or a salt, hydrate, deuterated analog, or fluorinated analog thereof.

In another aspect, methods are provided comprising administering an effective amount of compound 1, according to the formula:

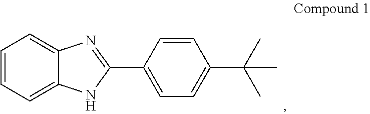 [SMILES: CC(C)(C)c1ccc(-c2nc3ccccc3[nH]2)cc1]

or a salt, hydrate, deuterated analog, or fluorinated analog thereof to a patient who has, or is at risk for developing, systemic immune activation, wherein the compound is not administered to treat a neurodegenerative disorder. In some embodiments, the patient does not have neuroinflammation. In some embodiments, the patient does not have clinically meaningfully neuroinflammation. In some embodiments, systemic immune activation does not comprise clinically significant neuroinflammation. In some embodiments, the patient has or is at risk for peripheral inflammation.

In a further aspect, methods are provided for treating a patient who has, or who is at risk for developing, sepsis, comprising:
administering to the patient an effective amount of a compound of Formula I:

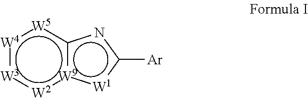

or a salt, hydrate, deuterated analog, or fluorinated analog thereof,
wherein:
Ar is

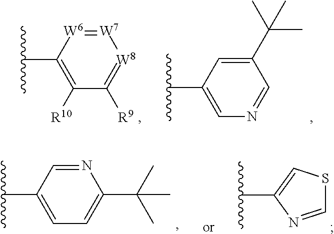

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

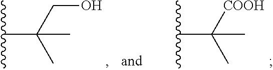

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

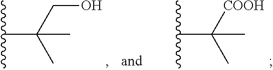

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

### 2.1. Pre-Treatment Symptoms and Signs

In the methods described herein, the patient has systemic immune activation or is at risk for systemic immune activation.

### 2.1.1. Systemic Inflammation

In some embodiments, the systemic immune activation is acute systemic inflammation. In some embodiments, the patient has or is at risk for acute peripheral inflammation. In some embodiments, the systemic immune activation is not chronic systemic inflammation. In some embodiments, the systemic immune activation is chronic systemic inflammation.

In some embodiments, systemic immune activation includes neuroinflammation. In particular embodiments, the compound of Formula I is not Compound 1 and the patient has a neurodegenerative disease. In specific embodiments, the patient has a motor neuron disease, amyotrophic lateral sclerosis (ALS), Alzheimer's disease, frontotemporal dementia, dementia with Lewy bodies, Parkinson's disease, Huntington's disease, a demyelinating disease, or multiple sclerosis.

In some embodiments, systemic immune activation does not include neuroinflammation. In some embodiments, systemic immune activation does not include clinically meaningfully neuroinflammation.

### 2.1.2. Pre-Treatment Serum CRP and IL-6 Levels

In some embodiments, the patient has elevated pre-treatment levels of serum C-reactive protein (CRP).

In some embodiments, the patient has a pre-treatment CRP level of at least 2 mg/L. In some embodiments, the patient has a pre-treatment CRP level of at least 5 mg/L. In some embodiments, the patient's pre-treatment CRP level is at least 2 mg/L, 2.5 mg/L, 3 mg/L, 3.5 mg/L, 4 mg/L, 4.5 mg/L, or 5 mg/L. In some embodiments, the patient has pre-treatment CRP levels of at least 7.5 mg/L, 10 mg/L, 12.5 mg/L, or 15 mg/L. In certain embodiments, the patient's pre-treatment CRP level is at least 7.5 mg/L. In certain embodiments, the patient has a pre-treatment CRP level of at least 10 mg/L. In certain embodiments, the patient has a pre-treatment CRP level of at least 12.5 mg/L. In certain embodiments, the patient has a pre-treatment CRP level of at least 15 mg/L. In certain preferred embodiments, the patient has a pre-treatment CRP level of at least 10 mg/L. In some embodiments, the patient has pre-treatment CRP levels of at least 20 mg/L, 25 mg/L, 30 mg/L, 35 mg/L, 40 mg/L, 45 mg/L, or 50 mg/L. In certain embodiments, the patient's pre-treatment CRP level is at least 20 mg/L. In certain embodiments, the patient has a pre-treatment CRP level of at least 25 mg/L. In certain embodiments, the patient has a pre-treatment CRP level of at least 30 mg/L. In certain embodiments, the patient has a pre-treatment CRP level of at least 35 mg/L. In certain embodiments, the patient's pre-treatment CRP level is at least 40 mg/L. In certain embodiments, the patient has a pre-treatment CRP level of at least 45 mg/L. In certain embodiments, the patient has a pre-treatment CRP level of at least 50 mg/L. In certain preferred embodiments, the patient has a pre-treatment CRP level of at least 40 mg/L.

In some embodiments of the methods described herein, the patient has elevated pre-treatment serum levels of IL-6.

In some embodiments, the patient has a pre-treatment serum IL-6 level of at least 2 pg/ml. In various embodiments, the patient has a pre-treatment serum IL-6 level of at least 2 pg/ml, at least 3 pg/ml, at least 4 pg/ml, at least 5 pg/ml, at least 6 pg/ml, at least 7 pg/ml, at least 8 pg/ml, at least 9 pg/ml, at least 10 pg/ml, at least 11 pg/ml, at least 12 pg/ml, at least 13 pg/ml, at least 14 pg/ml, or at least 15 pg/ml. In certain embodiments, the patient has a pre-treatment serum IL-6 level of at least 2.5 pg/ml. In certain embodiments, the patient has a pre-treatment serum IL-6 level of at least 4 pg/ml. In certain embodiments, the patient has a pre-treatment serum IL-6 level of at least 5 pg/ml. In certain embodiments, the patient has a pre-treatment serum IL-6 level of at least 7.5 pg/ml. In certain embodiments, the patient has a pre-treatment serum IL-6 level of at least 10 pg/ml. In certain embodiments, the patient has a pre-treatment serum IL-6 level of at least 12.5 pg/ml. In certain embodiments, the patient has a pre-treatment serum IL-6 level of at least 15 pg/ml. In some embodiments, the patient has a pre-treatment serum IL-6 level of at least 20 pg/ml. In various embodiments, the patient has a pre-treatment serum IL-6 level of at least 20 pg/ml, at least 30 pg/ml, at least 40 pg/ml, at least 50 pg/ml, at least 60 pg/ml, at least 70 pg/ml, at least 80 pg/ml, at least 90 pg/ml, at least 100 pg/ml, at least 150 pg/ml, or at least 200 pg/ml. In certain embodiments, the patient has a pre-treatment serum IL-6 level of at least 30 pg/ml. In certain embodiments, the patient has a pre-treatment serum IL-6 level of at least 40 pg/ml. In certain embodiments, the patient has a pre-treatment serum IL-6 level of at least 50 pg/ml. In certain embodiments, the patient has a pre-treatment serum IL-6 level of at least 75 pg/ml. In certain embodiments, the patient has a pre-treatment serum IL-6 level of at least 100 pg/ml. In certain embodiments, the patient has a pre-treatment serum IL-6 level of at least 150 pg/ml. In certain embodiments, the patient has a pre-treatment serum IL-6 level of at least 200 pg/ml.

In some embodiments, the patient has elevated pre-treatment serum levels of CRP and elevated pre-treatment IL-6 levels. In certain embodiments, the patient has a pre-treatment serum IL-6 level of at least 2 pg/ml and a pre-treatment CRP level of at least 2 mg/L. In certain embodiments, the patient has a pre-treatment serum IL-6 level of at least 2 pg/ml and a pre-treatment CRP level of at least 2.5 mg/L. In certain embodiments, the patient has a pre-treatment serum IL-6 level of at least 2 pg/ml and a pre-treatment CRP level of at least 5 mg/L. In certain embodiments, the patient has a pre-treatment serum IL-6 level of at least 2 pg/ml and a pre-treatment CRP level of at least 10 mg/L. In certain embodiments, the patient has a pre-treatment serum IL-6 level of at least 4 pg/ml and a pre-treatment CRP level of at least 2 mg/L. In certain embodiments, the patient has a pre-treatment serum IL-6 level of at least 4 pg/ml and a pre-treatment CRP level of at least 2.5 mg/L. In certain embodiments, the patient has a pre-treatment serum IL-6 level of at least 4 pg/ml and a pre-treatment CRP level of at least 5 mg/L. In certain embodiments, the patient has a pre-treatment serum IL-6 level of at least 4 pg/ml and a pre-treatment CRP level of at least 10 mg/L. In certain embodiments, the patient has a pre-treatment serum IL-6 level of at least 5 pg/ml and a pre-treatment CRP level of at least 2 mg/L. In certain embodiments, the patient has a pre-treatment serum IL-6 level of at least 5 pg/ml and a pre-treatment CRP level of at least 2.5 mg/L. In certain embodiments, the patient has a pre-treatment serum IL-6 level of at least 5 pg/ml and a pre-treatment CRP level of at least 5 mg/L. In certain embodiments, the patient has a pre-treatment serum IL-6 level of at least 5 pg/ml and a pre-treatment CRP level of at least 10 mg/L. In certain embodiments, the patient has a pre-treatment serum IL-6 level of at least 10 pg/ml and a pre-treatment CRP level of at least 2 mg/L. In certain embodiments, the patient has a pre-treatment serum IL-6 level of at least 10 pg/ml and a pre-treatment CRP level of at least 2.5 mg/L. In certain embodiments, the patient has a pre-treatment serum IL-6 level of at least 10 pg/ml and a pre-treatment CRP level of at least 5 mg/L. In certain embodiments, the patient has a pre-treatment serum IL-6 level of at least 10 pg/ml and a pre-treatment CRP level of at least 10 mg/L.

In some embodiments, the patient has a pre-treatment serum IL-6 level of at least 10 pg/ml and a pre-treatment CRP level of at least 10 mg/L. In certain embodiments, the patient has a pre-treatment serum IL-6 level of at least 10 pg/ml and a pre-treatment CRP level of at least 20 mg/L. In certain embodiments, the patient has a pre-treatment serum IL-6 level of at least 10 pg/ml and a pre-treatment CRP level of at least 30 mg/L. In certain embodiments, the patient has a pre-treatment serum IL-6 level of at least 10 pg/ml and a pre-treatment CRP level of at least 40 mg/L. In certain embodiments, the patient has a pre-treatment serum IL-6 level of at least 20 pg/ml and a pre-treatment CRP level of at least 10 mg/L. In certain embodiments, the patient has a pre-treatment serum IL-6 level of at least 20 pg/ml and a pre-treatment CRP level of at least 20 mg/L. In certain embodiments, the patient has a pre-treatment serum IL-6 level of at least 20 pg/ml and a pre-treatment CRP level of at least 30 mg/L. In certain embodiments, the patient has a pre-treatment serum IL-6 level of at least 20 pg/ml and a pre-treatment CRP level of at least 40 mg/L. In certain embodiments, the patient has a pre-treatment serum IL-6 level of at least 30 pg/ml and a pre-treatment CRP level of at least 10 mg/L. In certain embodiments, the patient has a pre-treatment serum IL-6 level of at least 30 pg/ml and a pre-treatment CRP level of at least 20 mg/L. In certain embodiments, the patient has a pre-treatment serum IL-6 level of at least 30 pg/ml and a pre-treatment CRP level of at least 30 mg/L. In certain embodiments, the patient has a pre-treatment serum IL-6 level of at least 30 pg/ml and a pre-treatment CRP level of at least 40 mg/L. In certain embodiments, the patient has a pre-treatment serum IL-6 level of at least 40 pg/ml and a pre-treatment CRP level of at least 10 mg/L. In certain embodiments, the patient has a pre-treatment serum IL-6 level of at least 40 pg/ml and a pre-treatment CRP level of at least 20 mg/L. In certain embodiments, the patient has a pre-treatment serum IL-6 level of at least 40 pg/ml and a pre-treatment CRP level of at least 30 mg/L. In certain embodiments, the patient has a pre-treatment serum IL-6 level of at least 40 pg/ml and a pre-treatment CRP level of at least 40 mg/L.

### 2.1.3. CRS

In some embodiments, the patient has cytokine release syndrome (CRS). In some embodiments, the patient is at risk for CRS. In some embodiments, the patient has infection by a virus, such as an influenza virus or a coronavirus.

### 2.1.4. Hypercoagulability

In some embodiments, the patient has confirmed or suspected hypercoagulability. Hypercoagulability has been shown to be linked to certain inflammatory conditions related to the pro-inflammatory cytokines IL-6, IL-1β, and/or IL-8 (see e.g., Bester and Pretorius, Effects of IL-1β, IL-6, and IL-8 on erythrocytes, platelets, and clot viscoelasticity. Scientific Reports (2016) 6:32188, pgs 1-10). Thus, patients who have increased amounts of pro-inflammatory cytokine IL-6 can induce hypercoagulability in the patient.

### 2.1.5. Confirmed or Suspected Viral Lung Infection

In various embodiments of the methods described herein, the patient has a confirmed or suspected viral infection. In some embodiments, the infection is by a virus selected from the group consisting of: coronavirus, influenza virus, rhinovirus, respiratory syncytial virus, metapneumovirus, adenovirus, and boca virus.

In certain embodiments, the virus is an influenza virus. In particular embodiments, the influenza virus is selected from the group consisting of: parainfluenza virus 1, parainfluenza virus 2, influenza A virus, and influenza B virus.

In certain embodiments, the virus is a coronavirus. In particular embodiments, the coronavirus is selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2. In some embodiments, the coronavirus is SARS-CoV-2.

In particular embodiments, the CRS is caused by infection with severe acute respiratory syndrome beta coronavirus (SARS-CoV) and the patient has severe acute respiratory syndrome (SARS). In particular embodiments, the CRS is caused by infection with middle eastern respiratory syndrome virus (MERS-CoV) and the patient has middle east respiratory syndrome (MERS). In particular embodiments, the CRS is caused by infection with SARS-CoV-2 virus and the patient has coronavirus disease 2019 (COVID-19).

In some embodiments, the patient is at risk for CRS.

In some embodiments, the patient has a hyperinflammatory response. In certain embodiments, the patient is at risk for a hyperinflammatory response. In some embodiments, methods include preventing a hyperinflammatory response in a patient who is at risk for CRS.

In certain embodiments, the patient at risk for CRS has been treated or is being treated with a checkpoint inhibitor, a bispecific T-cell engager, or CAR-T cells.

In certain embodiments, the patient at risk for CRS has a viral infection. In some embodiments, the infection is by a virus selected from the group consisting of: coronavirus, influenza virus, rhinovirus, respiratory syncytial virus, metapneumovirus, adenovirus, and boca virus.

In certain embodiments, the virus is a coronavirus. In particular embodiments, the coronavirus is selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2. In some embodiments, the coronavirus is SARS-CoV-2.

In particular embodiments, the patient at risk for CRS has severe acute respiratory syndrome (SARS). In particular embodiments, the patient at risk for CRS has middle eastern respiratory syndrome (MERS). In particular embodiments, the patient at risk for CRS has coronavirus disease 2019 (COVID-19).

In certain embodiments, the virus is an influenza virus. In particular embodiments, the virus is any one or combination of the following influenza viruses: parainfluenza virus 1, parainfluenza virus 2, influenza A virus, and influenza B virus.

In various embodiments, viral infection has been or is concomitantly confirmed by detection of viral genetic material in a fluid sample from the patient. In some embodiments, viral infection has not been or is not concomitantly confirmed by detection of viral genetic material in a fluid sample from the patient, but is presumed based on clinical presentation and history. In particular embodiments, treatment is initiated before confirmation by detection of viral genetic material. In specific embodiments, treatment is initiated before confirmation by detection of viral genetic material, and viral infection is later confirmed by detection of viral genetic material or virus-specific IgM and/or IgG in the patient's serum.

### 2.1.6. Sepsis

In some embodiments, the patient has sepsis. In some embodiments, the patient is at risk for developing sepsis.

### 2.1.7. Fever

In some embodiments, the patient has fever. In some embodiments, the patient has a body temperature greater than 37.5° C. In some embodiments, the body temperature is 37.6° C. or greater, 37.7° C. or greater, 37.8° C. or greater, 37.9° C. or greater, 38° C. or greater, 38.1° C. or greater, 38.2° C. or greater, 38.3° C. or greater, 38.4° C. or greater, 38.5° C. or greater, 38.6° C. or greater, 38.7° C. or greater, 38.8° C. or greater, 38.9° C. or greater, 39° C. or greater, 39.1° C. or greater, 39.2° C. or greater, 39.3° C. or greater, 39.4° C. or greater, 39.5° C. or greater, 39.6° C. or greater, 39.7° C. or greater, 39.8° C. or greater, 39.9° C. or greater, 40° C. or greater, 40.1° C. or greater, 40.2° C. or greater, 40.3° C. or greater, 40.4° C. or greater, 40.5° C. or greater, 40.6° C. or greater, 40.7° C. or greater, 40.8° C. or greater, 40.9° C. or greater, 41° C. or greater, or 42° C. or greater. In some embodiments, the patient has a body temperature greater than 37.5° C. for 24 hours or more, 48 hours or more, 72 hours or more, 96 hours or more, 5 days or more, 6 days or more, 1 week or more, 1.5 weeks or more, or 2 weeks or more. In typical embodiments, the body temperature is measured from clinically accessible measurement sites on the patient. In various embodiments, the measurement site is the patient's forehead, temple, and/or other external body surfaces. In some embodiments, the measurement site is the oral cavity, rectal cavity, axilla area, or tympanic membrane.

### 2.1.8. Reduced Blood Oxygen Saturation

In some embodiments, the patient has a blood oxygen saturation level (SpO₂) of less than 95%. In some embodiments, the patient has a blood oxygen saturation level (SpO₂) of less than 94%. In some embodiments, the patient has a blood oxygen saturation level (SpO₂) of 93% or less. In some embodiments, the patient has an SpO₂ level of 92% or less, 91% or less, 90% or less, 85% or less, 80% or less, 75% or less, 70% or less, 65% or less, 60% or less, 55% or less, 50% or less, 45% or less, 40% or less, 35% or less, 30% or less, or 25% or less. In some embodiments, the patient requires mechanical ventilation and/or supplemental oxygen.

### 2.1.9. Pneumonia

In some embodiments, the patient has pneumonia.

### 2.1.10. Hospitalization

In some embodiments, the patient is hospitalized.

### 2.1.11. Mechanical or Assisted Ventilation

In some embodiments, the patient is on a ventilator. In some embodiments, the patient is not on a ventilator.

### 2.1.12. Pre-Treatment d-Dimer and Sepsis-Induced Coagulopathy (SIC) Score

In certain embodiments, the patient has elevated pre-treatment levels of d-dimer above baseline (e.g. >1 μg/ml or elevated at or above the upper limit of normal). In certain embodiments, the patient has elevated pre-treatment levels of sepsis-induced coagulopathy (SIC) total score of 4 or more with total score of prothrombin time and coagulation exceeding 2, or a score at or above the upper limit of normal.

### 2.1.13. Pre-Treatment Lymphocyte Count

In some embodiments, the patient has a pre-treatment median lymphocyte count per mm³ of 1000 or less. In certain embodiments, the patient has a pre-treatment median lymphocyte count per mm³ of 950 or less. In certain embodiments, the patient has a pre-treatment median lymphocyte count per mm³ of 900 or less, 850 or less, 800 or less, 750 or less, 700 or less, 650 or less, or 600 or less. In particular embodiments, the patient has a pre-treatment median lymphocyte count per mm³ of 800. In particular embodiments, the patient has a pre-treatment median lymphocyte count per mm³ or 700.

### 2.1.14. Pre-Treatment Platelet Count

In some embodiments, the patient has a pre-treatment median platelet count per mm³ of 175,000 or less. In certain embodiments, the patient has a pre-treatment median platelet count per mm³ of 172,000 or less. In certain embodiments, the patient has a pre-treatment median platelet count per mm³ of 170,000 or less, 165,000 or less, 160,000 or less, 155,000 or less, 150,000 or less, 145,000 or less, 140,000 or less, 135,000 or less, 130,000 or less, 125,000 or less, 120,000 or less, 115,000 or less, 110,000 or less, 105,000 or less, or 100,000 or less. In particular embodiments, the patient has a pre-treatment median platelet count per mm³ of 160,000 or less. In particular embodiments, has a pre-treatment median platelet count per mm³ or 140,000 or less.

### 2.1.15. Pre-Treatment Neutrophil-to-Lymphocyte Ratio

In some embodiments, the patient has a pre-treatment neutrophil-to-lymphocyte ratio (NLR) greater than 2.0. In some embodiments, the patient has a pre-treatment NLR greater than 3.0. In some embodiments, the patient has a pre-treatment NLR greater than 2.8, 2.9, 3.0, 3.1, 3.2, 3.3, 3.4, 3.5, 3.6, 3.7, 3.8, or 3.9. In some embodiment, the patient has a pre-treatment NLR greater than 4.0.

### 2.1.16. Pre-Treatment Lactate Dehydrogenase

In some embodiments, the patient has elevated pre-treatment levels of lactate dehydrogenase at or above baseline (e.g. 250 units/liter, or elevated at or above the upper limit of normal). In some embodiments, the patient has elevated pre-treatment levels of lactate dehydrogenase at or above 250 units/liter, 300 units/liter, 350 units/liter, 350 units/liter, 400 units/liter, 450 units/liter, or 500 units/liter.

### 2.2. Patient Age

In some embodiments, the patient is older than 60 years old. In some embodiments, the patient is older than 50 years old. In some embodiments, the patient is older than 50, 51, 52, 53, 54, 55, 56, 57, 58, 59, or 60 years old. In some embodiments, the patient is younger than 60, 59, 58, 57, 56, 55, 54, 53, 52, 51, or 50 years old. In some embodiments the patient is a young adult between the age of 20-35. In some embodiments, the patient is middle aged, between the age of 35-50. In some embodiments, the patient is a teenager between the age of 13-19. In some embodiments, the patient is a child between the age of 5-12. In alternative embodiments, the patient is a toddler between the age of 1-4. In further embodiments, the patient is an infant between the age of newborn to one year old.

### 2.3. Compounds

In some embodiments, the compound of the methods described herein is a compound of Formula I:

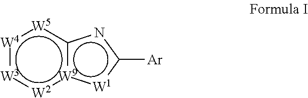

or a salt, hydrate, deuterated analog, or fluorinated analog thereof,
wherein:
Ar is

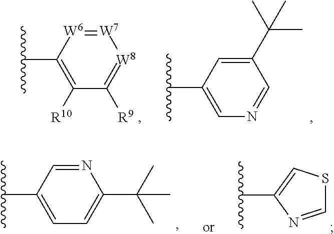

W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(—O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy,

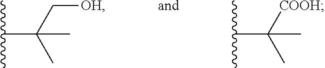

R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

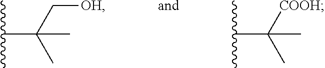

R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxoalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

In some embodiments, the compound of Formula I is selected from:

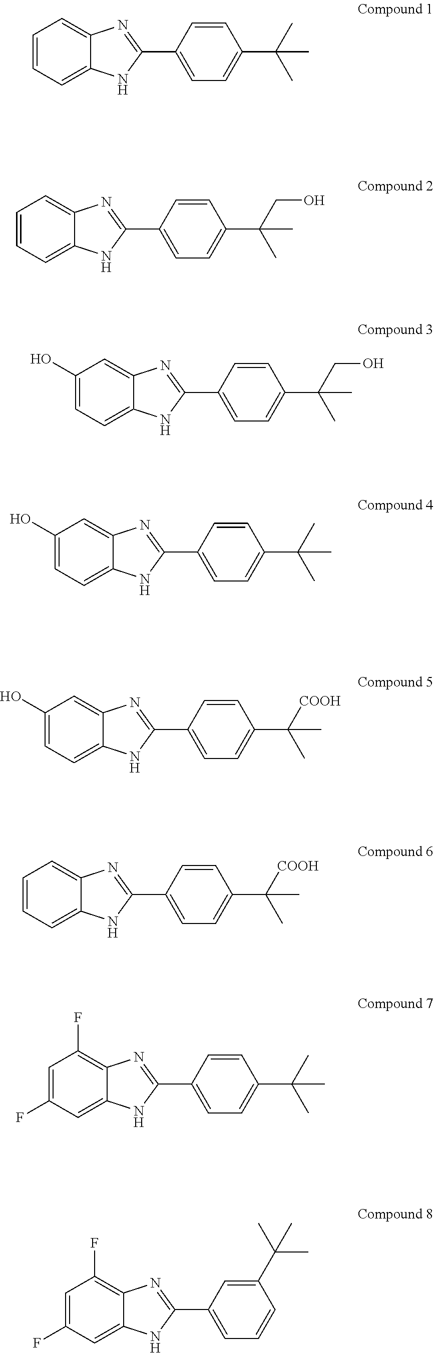

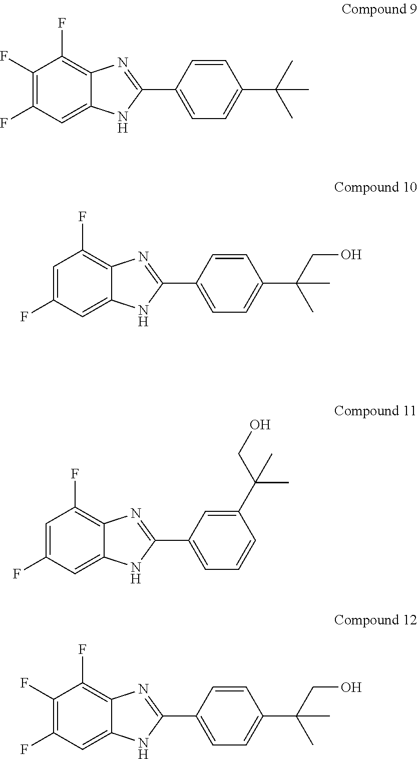 [SMILES: CC(C)(C)c1ccc(-c2nc3c(F)c(F)c(F)cc3[nH]2)cc1 ; CC(C)(CO)c1ccc(-c2nc3c(F)cc(F)cc3[nH]2)cc1 ; CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]

or a salt, hydrate, deuterated analog, or fluorinated analog thereof.

In particular embodiments, the compound of Formula I is compound 1, according to the formula:

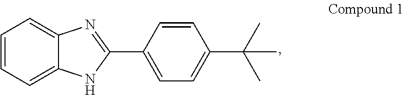 [SMILES: CC(C)(C)c1ccc(-c2nc3ccccc3[nH]2)cc1]

or a salt, hydrate, deuterated analog, or fluorinated analog thereof.

In particular embodiments, the compound of Formula I is compound 8, according to the formula:

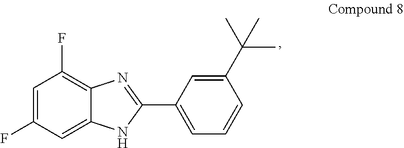 [SMILES: CC(C)(C)c1cccc(-c2nc3c(F)cc(F)cc3[nH]2)c1]

or a salt, hydrate, deuterated analog, or fluorinated analog thereof.

In particular embodiments, the compound of Formula I is 7, according to the formula:

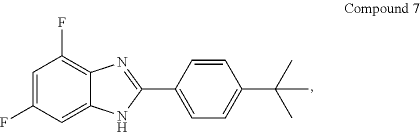 [SMILES: CC(C)(C)c1ccc(-c2nc3c(F)cc(F)cc3[nH]2)cc1]

or a salt, hydrate, deuterated analog, or fluorinated analog thereof.

In particular embodiments, the compound of Formula I is compound 9, according to the formula:

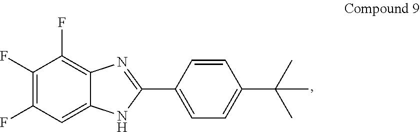 [SMILES: CC(C)(C)c1ccc(-c2nc3c(F)c(F)c(F)cc3[nH]2)cc1]

or a salt, hydrate, deuterated analog, or fluorinated analog thereof.

### 2.4. Pharmaceutical Composition

The compound of Formula I used in the methods described herein can be formulated in any appropriate pharmaceutical composition for administration by any suitable route of administration. Suitable routes of administration include, but are not limited to, intravenous and oral routes of administration. Suitable routes also include pulmonary administration, including by oral inhalation. The most suitable route may depend upon the condition and disorder of the recipient. The formulations may conveniently be presented in unit dosage form and may be prepared by any of the methods known in the art of pharmacy.

All methods include the step of bringing into association a compound of Formula I, or a salt, hydrate, deuterated analog, or fluorinated analog thereof (“active ingredient”), with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein is parental administration. In certain embodiments, the route of administration for use in the methods described herein is intravenous administration. In certain embodiments, the route of administration for use in the methods described herein is oral administration.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, 8th Revised Ed. (2017), incorporated by reference in its entirety.

The term “pharmaceutically acceptable salt” refers to salts prepared from pharmaceutically acceptable non-toxic acids or bases including inorganic acids and bases and organic acids and bases. When the compounds of the present methods described herein are basic, salts may be prepared from pharmaceutically acceptable non-toxic acids including inorganic and organic acids. Suitable pharmaceutically acceptable acid addition salts for the compounds of the methods described herein include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, malefic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

### 2.5. Dosage Regimens

The compound of formula I is administered at a dose sufficient to treat or prevent systemic immune activation, such as CRS, sepsis, or for compounds other than Compound 1, neuroinflammation.

In various embodiments, the compound of Formula I is administered using a weight based dose. In some embodiments, the compound of Formula I is administered in an amount of at least 0.5 mg/kg. In certain embodiments, the compound of Formula I is administered orally in an amount of at least 1 mg/kg. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg.

In various embodiments, the dose of the compound of Formula I is at least 10 mg/kg. In certain embodiments, the dose is at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 150 mg/kg, at least 175 mg/kg, or at least 200 mg/kg. In certain embodiments, the dose is 250 mg/kg, 300 mg/kg, 350 mg/kg, 400 mg/kg, 450 mg/kg, 500 mg/kg, 600 mg/kg, 650 mg/kg, 700 mg/kg, 750 mg/kg, 800 mg/kg, 850 mg/kg, 900 mg/kg, 950 mg/kg, or 1000 mg/kg. In certain embodiments, the dose is 0.5 mg/kg to 100 mg/kg per day. In certain embodiments, the dose is 2 mg/kg to 100 mg/kg per day. In certain embodiments, the dose is 25 mg/kg to 1000 mg/kg per day.

In various embodiments, the dose of the compound of Formula I is 25 mg/kg. In certain embodiments, the dose is at least 25 mg/kg. In certain embodiments, the dose is at least 50 mg/kg, at least 100 mg/kg, at least 150 mg/kg, at least 175 mg/kg, or at least 200 mg/kg. In certain embodiments, the dose is 250 mg/kg, 500 mg/kg, 750 mg/kg, or 1000 mg/kg. In certain embodiments, the dose is 25 mg/kg to 1,000 mg/kg per day.

In some embodiments, the compound of Formula I is administered at a dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg or 0.1 mg/kg. In some embodiments, the compound of Formula I is administered at a dose of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. In some embodiments, the compound of Formula I is administered at a dose of 1.5 mg/kg, 2 mg/kg, 2.5 mg/kg, 3 mg/kg, 3.5 mg/kg, 4 mg/kg, 4.5 mg/kg, or 5 mg/kg. In some embodiments, the compound of Formula I is administered at a dose of 6 mg/kg, 7 mg/kg, 8 mg/kg, 9 mg/kg, 10 mg/kg, 12 mg/kg, 15 mg/kg, 20 mg/kg, 30 mg/kg, 40 mg/kg, or 50 mg/kg. In some embodiments, the compound of Formula I is administered at a dose of 10 mg/kg, 50 mg/kg, 8 mg/kg, 100 mg/kg, 150 mg/kg, 200 mg/kg, 250 mg/kg, 300 mg/kg, 350 mg/kg, 400 mg/kg, or 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg.

In some embodiments, the compound of formula (I) is administered in a dose that is independent of patient weight or surface area (flat dose).

In some embodiments, the flat dose is 0.1 mg, 0.2 mg, 0.3 mg, 0.4 mg, 0.5 mg, 0.6 mg, 0.7 mg, 0.8 mg, 0.9 mg, or 1 mg. In some embodiments, the flat dose is 1 mg, 2 mg, 3 mg, 4 mg, 5 mg, 6 mg, 7 mg, 8 mg, 9 mg, or 10 mg. In some embodiments, the flat dose is 11 mg, 12 mg, 13 mg, 14 mg, 15 mg, 16 mg, 17 mg, 18 mg, 19 mg, or 20 mg. In some embodiments, the flat dose is 25 mg, 30 mg, 40 mg, or 50 mg. In some embodiments, the flat dose is 60 mg, 70 mg, 80 mg, 90 mg, or 100 mg. In some embodiments, the flat dose is 200 mg, 300 mg, 400 mg, 500 mg, 600 mg, 700 mg, 800 mg, 900 mg, or 1000 mg. In some embodiments, the flat dose is 0.1-1 mg, 1-10 mg, 10-15 mg, 15-20 mg, 20-30 mg, 30-40 mg, or 40-50 mg. In some embodiments, the flat dose is 1-50 mg, 50-100 mg, 100 mg-200 mg, 200 mg-300 mg, 300 mg-400 mg, 400 mg-500 mg, 500 mg-600 mg, 600 mg-700 mg, 700 mg-800 mg, 800 mg-900 mg, or 900 mg-1000 mg.

In various embodiments, the dose is 10-5000 mg. In certain embodiments, the dose is 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the dose is 1500 mg, 2000 mg, 2500 mg, 3000 mg, 3500 mg, 4000 mg, 4500 mg, or 5000 mg.

In various embodiments, the dose is 25-2000 mg. In certain embodiments, the dose is 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg.

The compound of Formula I can be administered in a single dose or in multiple doses. In various intravenous embodiments, the compound of Formula I is administered once a day, once every 2 days, once every 3 days, once every 4 days, once every 5 days, once every 6 days, once every 7 days, once every 14 days, once every 21 days, once every 28 days, or once a month. In various intravenous embodiments, the compound of Formula I is administered twice a day, twice every 2 days, twice every 3 days, twice every 4 days, twice every 5 days, twice every 6 days, twice every 7 days, twice every 14 days, twice every 21 days, twice every 28 days, or twice a month.

### 2.6. Additional Agents

In some embodiments, the methods of the present disclosure include administering an effective amount of at least one second therapeutic agent.

### 2.6.1. Anti-Viral Agents

In some embodiments, the second therapeutic agent is an anti-viral agent, and the method of the present disclosure further comprises administering an effective amount of an anti-viral agent.

In particular embodiments, the anti-viral agent is selected from the group consisting of: favipiravir, remdesivir, and a combination of lopinavir and ritonavir.

In particular embodiments, the anti-viral agent is favipiravir.

In particular embodiments, the anti-viral agent is remdesivir.

In particular embodiments, the anti-viral agent is a combination of lopinavir and ritonavir.

### 2.6.2. Antibacterial Agents

In some embodiments, the method of the present disclosure further comprises administering an antibacterial agent. In some embodiments, the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin. In certain embodiments, the antibacterial agent is azithromycin.

### 2.6.3. Angiotensin Receptor Blocker (ARB)

In some embodiments, the methods comprise administering a second therapeutic agent. In particular embodiments, the second therapeutic agent is an ARB, and the method of the present disclosure further comprises administering an effective amount of an ARB.

In particular embodiments, the ARB is selected from losartan, valsartan, azilsartan, candesartan, eprosartan, irgesartan, olmesartan, and telmisartan.

### 2.6.4. IL-6 Antagonists

In certain embodiments, the patient is further administered an IL-6 antagonist. In some embodiments, the IL-6 inhibitor or antagonist is selected from the group consisting of: an anti-IL-6 receptor antibody or an antigen binding fragment thereof; an anti-IL-6 antibody or an antigen binding fragment thereof; and a JAK/STAT inhibitor.

### 2.6.4.1. Anti-IL-6 Receptor Antibodies

In various embodiments, the IL-6 antagonist is an anti-IL-6 receptor (anti-IL-6R) antibody or antigen-binding fragment or derivative thereof.

In typical embodiments, the anti-IL-6R reduces the biological activity of IL-6 receptor.

In some embodiments, the IL-6 antagonist is an anti-IL-6R monoclonal antibody. In some embodiments, the IL-6 antagonist is a polyclonal composition comprising a plurality of species of anti-IL-6R antibodies, each of the plurality having unique CDRs.

In some embodiments, the anti-IL-6R antibody is a Fab, Fab′, F(ab′)2, Fv, scFv, (scFv)2, single chain antibody molecule, dual variable domain antibody, single variable domain antibody, linear antibody, or V domain antibody.

In some embodiments, the anti-IL-6R antibody comprises a scaffold. In certain embodiments, the scaffold is Fc, optionally human Fc. In some embodiments, the anti-IL-6R antibody comprises a heavy chain constant region of a class selected from IgG, IgA, IgD, IgE, and IgM. In certain embodiments, the anti-IL-6R antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6R antigen-binding fragment.

In some embodiments, the antibody is bispecific or multispecific, with at least one of the antigen-binding portions having specificity for IL-6 receptor.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6R antibody has a KD for binding human IL-6 receptor of less than 100 nM. In some embodiments, the anti-IL-6R antibody has a KD for binding human IL-6 receptor of less than 75 nM, 50 nM, 25 nM, 20 nM, 15 nM, or 10 nM. In particular embodiments, the anti-IL-6R antibody has a KD for binding human IL-6 receptor of less than 5 nM, 4 nM, 3 nM, or 2 nM. In selected embodiments, the anti-IL-6R antibody has a KD for binding human IL-6 receptor of less than 1 nM, 750 pM, or 500 pM. In specific embodiments, the anti-IL-6R antibody has a KD for binding human IL-6 receptor of no more than 500 pM, 400 pM, 300 pM, 200 pM, or 100 pM.

In typical embodiments, the anti-IL-6R antibody has an elimination half-life following intravenous administration of at least 7 days. In certain embodiments, the anti-IL-6R antibody has an elimination half-life of at least 14 days, at least 21 days, or at least 30 days.

In some embodiments, the anti-IL-6R antibody has a human IgG constant region with at least one amino acid substitution that extends serum half-life as compared to the unsubstituted human IgG constant domain.

### Tocilizumab and Derivatives

In certain embodiments, the anti-IL-6R antibody or antigen-binding portion thereof comprises all six CDRs of tocilizumab. In particular embodiments, the antibody or antigen-binding portion thereof comprises the tocilizumab heavy chain V region and light chain V region. In specific embodiments, the antibody is the full-length tocilizumab antibody.

In various embodiments, the anti-IL-6R antibody is a derivative of tocilizumab.

In some embodiments, the tocilizumab derivative includes one or more amino acid substitutions in the tocilizumab heavy and/or light chain V regions.

In certain embodiments, the tocilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the tocilizumab anti-IL-6R antibody, while retaining specificity for human IL-6 receptor.

In certain embodiments, the tocilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of tocilizumab. The percent sequence identity is determined using BLAST algorithms using default parameters.

In certain embodiments, the tocilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of tocilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

### Sarilumab and Derivatives

In certain embodiments, the anti-IL-6R antibody or antigen-binding portion thereof comprises all six CDRs of sarilumab. In particular embodiments, the antibody or antigen-binding portion thereof comprises the sarilumab heavy chain V region and light chain V region. In specific embodiments, the antibody is the full-length sarilumab antibody.

In various embodiments, the anti-IL-6R antibody is a derivative of sarilumab.

In some embodiments, the sarilumab derivative includes one or more amino acid substitutions in the sarilumab heavy and/or light chain V regions.

In certain embodiments, the sarilumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the sarilumab anti-IL-6R antibody, while retaining specificity for human IL-6 receptor.

In certain embodiments, the sarilumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of sarilumab. The percent sequence identity is determined using BLAST algorithms using default parameters.

In certain embodiments, the sarilumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sarilumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

### Vobarilizumab and Derivatives

In certain embodiments, the anti-IL-6R antibody or antigen-binding portion thereof comprises all six CDRs of vobarilizumab. In particular embodiments, the antibody or antigen-binding portion thereof comprises the vobarilizumab heavy chain V region and light chain V region. In specific embodiments, the antibody is the full-length vobarilizumab antibody.

In various embodiments, the anti-IL-6R antibody is a derivative of vobarilizumab.

In some embodiments, the vobarilizumab derivative includes one or more amino acid substitutions in the vobarilizumab heavy and/or light chain V regions.

In certain embodiments, the vobarilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the vobarilizumab anti-IL-6R antibody, while retaining specificity for human IL-6 receptor.

In certain embodiments, the vobarilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of vobarilizumab. The percent sequence identity is determined using BLAST algorithms using default parameters.

In certain embodiments, the vobarilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of vobarilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

### Other Anti-IL-6R Antibodies and Derivatives

In certain embodiments, the anti-IL-6R antibody or antigen-binding portion thereof comprises all six CDRs of an antibody selected from the group consisting of: SA237 (Roche), NI-1201 (NovImmune), and an antibody described in US 2012/0225060. In particular embodiments, the antibody or antigen-binding portion thereof comprises the heavy chain V region and light chain V region of an antibody selected from the group consisting of: SA237 (Roche), NI-1201 (NovImmune), and an antibody described in US 2012/0225060. In specific embodiments, the antibody is a full-length selected from the group consisting of: SA237 (Roche), NI-1201 (NovImmune), and an antibody described in US 2012/0225060.

In various embodiments, the anti-IL-6R antibody is a derivative of an antibody selected from the group consisting of: SA237 (Roche), NI-1201 (NovImmune), or an antibody described in US 2012/0225060.

### Anti-IL-6:IL-6R Complex Antibodies

In various embodiments, the IL-6 antagonist is an antibody specific for the complex of IL-6 and IL-6R. In certain embodiments, the antibody has the six CDRs of an antibody selected from those described in US 2011/0002936, which is incorporated herein by reference in its entirety.

### 2.6.4.2. Anti-IL-6 Antibodies

In various embodiments, the IL-6 antagonist is an anti-IL-6 antibody or antigen-binding fragment thereof.

In typical embodiments, the anti-IL-6 antibody or antigen-binding fragment thereof neutralizes the biological activity of human IL-6. In some embodiments, the neutralizing antibody prevents binding of IL-6 to the IL-6 receptor. In certain embodiments, the neutralizing antibody prevents binding of IL-6 to the soluble IL-6 receptor. In certain embodiments, the neutralizing antibody prevents binding of IL-6 to the membrane-bound IL-6 receptor. In certain embodiments, the neutralizing antibody prevents binding of IL-6 to both the soluble IL-6 receptor and the membrane-bound IL-6 receptor.

In some embodiments, the IL-6 antagonist is an anti-IL-6 monoclonal antibody. In some embodiments, the IL-6 antagonist is a polyclonal composition comprising a plurality of species of anti-IL-6 antibodies, each of the plurality having unique CDRs.

In some embodiments, the anti-IL-6 antibody is selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza). In some embodiments, the antigen-binding fragment is a fragment of an antibody selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

### 2.6.4.3. IL-6 Antagonist Peptides

In various embodiments, the IL-6 antagonist is an antagonist peptide.

In certain embodiments, the IL-6 antagonist is C326 (an IL-6 inhibitor by Avidia, also known as AMG220), or FE301, a recombinant protein inhibitor of IL-6 (Ferring International Center S.A., Conaris Research Institute AG). In some embodiments, the anti-IL-6 antagonist comprises soluble gp130, FE301 (Conaris/Ferring).

### 2.6.4.4. JAK and STAT Inhibitors

In various embodiments, the IL-6 antagonist is an inhibitor of the JAK signaling pathway. In some embodiments, the JAK inhibitor is a JAK1-specific inhibitor. In some embodiments, the JAK inhibitor is a JAK3-specific inhibitor. In some embodiments, the JAK inhibitor is a pan-JAK inhibitor. In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, momelotinib, INCB-039110, ABT-494, INCB-047986 and AC-410.

In various embodiments, the IL-6 antagonist is a STAT3 inhibitor. In a specific embodiment, the inhibitor is AZD9150 (AstraZeneca, Isis Pharmaceuticals), a STAT3 antisense molecule.

In typical embodiments, small molecule JAK inhibitors and STAT inhibitors are administered orally.

In various embodiments, the inhibitor is administered once or twice a day at an oral dose of 0.1-1 mg, 1-10 mg, 10-20 mg, 20-30 mg, 30-40 mg, or 40-50 mg. In some embodiments, the inhibitor is administered once or twice a day at a dose of 50-60 mg, 60-70 mg, 70-80 mg, 80-90 mg, or 90-100 mg. In some embodiments, the inhibitor is administered at a dose of 0.1, 0.5, 1, 2, 5, 10, 15, 20, 25, 30, 35, 40, 45, or 50 mg PO once or twice a day. In some embodiments, the inhibitor is administered at a dose of 75 mg or 100 mg PO once or twice a day.

### 2.6.5. Hydroxychloroquine and Chloroquine

In some embodiments, the second therapeutic agent is an anti-malarial agent, and the method of the present disclosure further comprises administering an effective amount of an anti-malarial agent.

In particular embodiments, the anti-malarial agent is hydroxychloroquine. In some embodiments, the anti-malarial agent is chloroquine.

### 2.6.6. COVID-19 Immune Serum or Plasma

In some embodiments, the second therapeutic agent is a COVID-19 immune serum or plasma, and the method of the present disclosure further comprises administering an effective amount of COVID-19 immune serum or plasma.

### 2.7. Post-Treatment Endpoints

In the methods described herein, following treatment (post-treatment) with a compound of Formula I, the patient has a reduction in one or more signs and/or symptoms of CRS.

### 2.8. Neutrophil Level

### 2.8.1. Absolute Neutrophil Count (ANC)

In the methods described herein, the compound of Formula I is administered at a dose that does not cause immune suppression.

In some embodiments, the immune suppression of the patient is measured by Absolute Neutrophil Count (ANC).

In some embodiments, the post-treatment ANC is at least 300 cells/μL. In various embodiments, the post-treatment ANC is at least 500 cells/μL, 600 cells/μL, 700 cells/μL, 800 cells/μL, 900 cells/μL, 1000 cells/μL, 1100 cells/μL, 1200 cells/μL, 1300 cells/μL, 1400 cells/μL, 1500 cells/μL, 1600 cells/μL, 1700 cells/μL, 1800 cells/μL, 1900 cells/μL, or 2000 cells/μL. In certain embodiments, the post-treatment ANC is at least 500 cells/μL. In certain embodiments, the post-treatment ANC is at least 750 cells/μL. In certain embodiments, the post-treatment ANC is at least 1000 cells/μL. In certain embodiments, the post-treatment ANC is at least 1250 cells/μL. In certain embodiments, the post-treatment ANC is at least 1500 cells/μL. In certain embodiments, the post-treatment ANC is at least 1750 cells/μL. In certain embodiments, the post-treatment ANC is at least 2000 cells/μL.

In some embodiments, the ANC is decreased by no more than 2500 cells/μL as compared to pre-treatment levels. In various embodiments, the ANC is decreased by no more than 2000 cells/μL, 1900 cells/μL, 1800 cells/μL, 1700 cells/μL, 1600 cells/μL, 1500 cells/μL, 1400 cells/μL, 1300 cells/μL, 1200 cells/μL, 1100 cells/μL, 1000 cells/μL, 900 cells/μL, 800 cells/μL, 700 cells/μL, 600 cells/μL, or 500 cells/μL, as compared to pre-treatment levels. In certain embodiments, the ANC is decreased by no more than 2000 cells/μL as compared to pre-treatment levels. In certain embodiments, the ANC is decreased by no more than 1750 cells/μL as compared to pre-treatment levels. In certain embodiments, the ANC is decreased by no more than 1500 cells/μL as compared to pre-treatment levels. In certain embodiments, the ANC is decreased by no more than 1250 cells/μL as compared to pre-treatment levels. In certain embodiments, the ANC is decreased by no more than 1000 cells/μL as compared to pre-treatment levels. In certain embodiments, the ANC is decreased by no more than 750 cells/μL as compared to pre-treatment levels. In certain embodiments, the ANC is decreased by no more than 500 cells/μL as compared to pre-treatment levels.

In some embodiments, the ANC is decreased by no more than 70% as compared to pre-treatment levels. In various embodiments, the ANC is decreased by no more than 60%, 50%, 40%, 30%, 20%, 10%, or 5% as compared to pre-treatment levels. In certain embodiments, the ANC is decreased by no more than 60% as compared to pre-treatment levels. In certain embodiments, the ANC is decreased by no more than 50% as compared to pre-treatment levels. In certain embodiments, the ANC is decreased by no more than 40% as compared to pre-treatment levels. In certain embodiments, the ANC is decreased by no more than 30% as compared to pre-treatment levels. In certain embodiments, the ANC is decreased by no more than 20% as compared to pre-treatment levels. In certain embodiments, the ANC is decreased by no more than 10% as compared to pre-treatment levels. In certain embodiments, the ANC is decreased by no more than 5% as compared to pre-treatment levels.

In some embodiments, the ANC is not decreased as compared to pre-treatment levels.

### 2.9. Reduction of IL-6 and C—Reactive Protein (CRP)

In typical embodiments, the administration of an effective amount of the compound of Formula I reduces the patient's serum IL-6 levels below pre-treatment levels.

In some embodiments, the serum IL-6 level is decreased by at least 10% as compared to pre-treatment levels. In various embodiments, the serum IL-6 level is decreased by at least 20%, 30%, 40%, 50%, 60%, 70%, 80%, or 90% as compared to pre-treatment levels. In certain embodiments, the serum IL-6 level is decreased by at least 20% as compared to pre-treatment levels. In certain embodiments, the serum IL-6 level is decreased by at least 30% as compared to pre-treatment levels. In certain embodiments, the serum IL-6 level is decreased by at least 40% as compared to pre-treatment levels. In certain embodiments, the serum IL-6 level is decreased by at least 50% as compared to pre-treatment levels. In certain embodiments, the serum IL-6 level is decreased by at least 60% as compared to pre-treatment levels. In certain embodiments, the serum IL-6 level is decreased by at least 70% as compared to pre-treatment levels. In certain embodiments, the serum IL-6 level is decreased by at least 80% as compared to pre-treatment levels. In certain embodiments, the serum IL-6 level is decreased by at least 90% as compared to pre-treatment levels.

In some embodiments, the administration of an effective amount of the compound of Formula I reduces the patient's serum CRP levels below pre-treatment levels.

In some embodiments, the post-treatment CRP level is no more than 45 mg/L. In certain embodiments, the post-treatment CRP level is no more than 40 mg/L. In certain embodiments, the post-treatment CRP level is no more than 30 mg/L. In certain embodiments, the post-treatment CRP level is no more than 20 mg/L. In certain embodiments, the post-treatment CRP level is no more than 10 mg/L. In certain embodiments, the post-treatment CRP level is no more than 5 mg/L. In certain embodiments, the post-treatment CRP level is no more than 2.5 mg/L. In certain embodiments, the post-treatment CRP level is no more than 2 mg/L. In certain embodiments, the post-treatment CRP level is no more than 1 mg/L.

In some embodiments, the CRP level is decreased by at least 10% as compared to pre-treatment levels. In various embodiments, the CRP level is decreased by at least 20%, 30%, 40%, 50%, 60%, 70%, 80%, or 90% as compared to pre-treatment levels. In certain embodiments, the CRP level is decreased by at least 20% as compared to pre-treatment levels. In certain embodiments, the CRP level is decreased by at least 30% as compared to pre-treatment levels. In certain embodiments, the CRP level is decreased by at least 40% as compared to pre-treatment levels. In certain embodiments, the CRP level is decreased by at least 50% as compared to pre-treatment levels. In certain embodiments, the CRP level is decreased by at least 60% as compared to pre-treatment levels. In certain embodiments, the CRP level is decreased by at least 70% as compared to pre-treatment levels. In certain embodiments, the CRP level is decreased by at least 80% as compared to pre-treatment levels. In certain embodiments, the CRP level is decreased by at least 90% as compared to pre-treatment levels.

### 2.10. Reduction of TNFα

In typical embodiments, the administration of an effective amount of the compound of Formula I reduces the patient's serum TNFα levels below pre-treatment levels.

In some embodiments, the serum TNFα level is decreased by at least 10% as compared to pre-treatment levels. In various embodiments, the serum TNFα level is decreased by at least 20%, 30%, 40%, 50%, 60%, 70%, 80%, or 90% as compared to pre-treatment levels. In certain embodiments, the serum TNFα level is decreased by at least 20% as compared to pre-treatment levels. In certain embodiments, the serum TNFα level is decreased by at least 30% as compared to pre-treatment levels. In certain embodiments, the serum TNFα level is decreased by at least 40% as compared to pre-treatment levels. In certain embodiments, the serum TNFα level is decreased by at least 50% as compared to pre-treatment levels. In certain embodiments, the serum TNFα level is decreased by at least 60% as compared to pre-treatment levels. In certain embodiments, the serum TNFα level is decreased by at least 70% as compared to pre-treatment levels. In certain embodiments, the serum TNFα level is decreased by at least 80% as compared to pre-treatment levels. In certain embodiments, the serum TNFα level is decreased by at least 90% as compared to pre-treatment levels.

### 2.11. Other Post-Treatment Endpoints

In some embodiments, administering an effective amount of a compound of Formula I to the patient at risk for CRS prevents a hyperinflammatory response in the patient.

In some embodiments, administering an effective amount of a compound of Formula I to the patient who has or is at risk of CRS results in a reduction in body temperature. In some embodiments, the patient, post-treatment with an effective amount of a compound of Formula I, has a body temperature of 37.5° C. or below. In some embodiments, the patient, post-treatment with an effective amount of a compound of Formula I, has a body temperature ranging from of 36 to 37.5° C.

In some embodiments, administering an effective amount of a compound of Formula I to the patient who has or is at risk of CRS results in a reduction in the risk of respiratory morbidity and mortality.

In some embodiments, administering an effective amount of a compound of Formula I to the patient who has or is at risk of CRS results in a reduction in the patient's need for supplemental oxygen. In some embodiments, administering an effective amount of a compound of Formula I to the patient who has or is at risk of CRS results in eliminating the patient's need for assisted ventilation.

Additionally, any of the primary and/or secondary endpoints relevant to CRS can be met by administering an effective amount of a compound of Formula I as described herein.

### 3. Examples

While various specific embodiments have been illustrated and described, the above specification is not restrictive. It will be appreciated that various changes can be made without departing from the spirit and scope of the invention(s). Many variations will become apparent to those skilled in the art upon review of this specification.

### 3.1.1. Example 1—TQS-168 Induces PGC-1a Protein Expression in a Murine Myeloid Cell Line In Vitro at 20 μM

### Study Methodology

Frozen BV2 murine microglia cells were thawed and propagated in complete media (RPMI, 10% heat-inactivated FBS, 1% L-glutamine, 1% Pen-Strep) until the growth rate reached log phase.

For protein expression analysis, DMSO (1:1000 final dilution) or TQS-168 in DMSO (1:1000 final dilution) at 20 μM was added to cell cultures. After 24 hours stimulation, supernatant was discarded and cell lysis buffer (Cell Signal) was added to adherent cells to extract protein. Total protein was quantified and normalized across all samples by BCA assays.

Two replicates of untreated cultures and three replicates of TQS-168-treated cultures were investigated, with each replicate containing lysates from 5,000,000 BV2 myeloid cells.

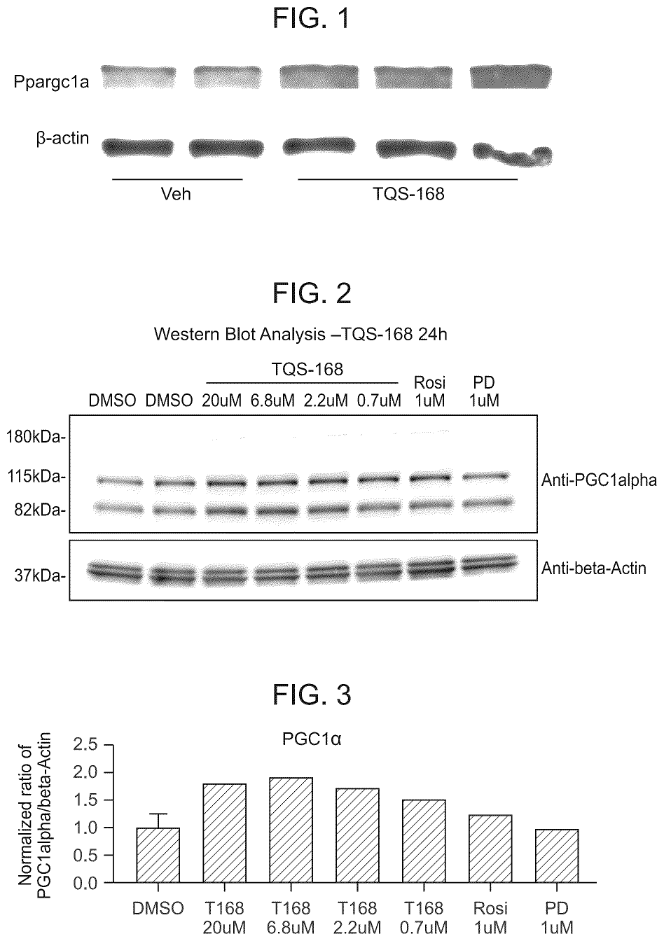

PGC1α expression was detected by Western Blot using an anti-PGC-1α antibody (SC13067, Santa Cruz Biotechnology, 1:500 dilution). Anti-β-actin antibody (SC8432, Santa Cruz Biotechnology, 1:2000 dilution) was used to quantify beta-actin, a housekeeping gene whose expression level is not know to be affected by TQS-168. A representative Western blot is shown in FIG. 1.

As shown, TQS-168 induces PGC-1α protein expression in murine BV2 microglia cells at 20 μM in vitro.

### 3.1.2. Example 2—TQS-168 Induces PGC-1α Protein Expression in a Murine Myeloid Cell Line In Vitro at Concentrations of 0.7 to 20 μM

For protein expression analysis, DMSO (1:1000 final dilution) or TQS-168 in DMSO (1:1000 final dilution) at 20 μM, 6.8 μM, 2.2 μM, and 0.7 μM were added to BV2 cell cultures. After 24 hours stimulation, supernatant was discarded and cell lysis buffer (Cell Signal) was added to adherent cells to extract protein. Total protein was quantified and normalized across all samples by BCA assays. PGC-1α was subsequently detected by Western Blot using an anti-PGC-1α antibody (SC13067, Santa Cruz Biotechnology, 1:500 dilution). β-actin was detected with an anti-β-actin antibody (SC8432, Santa Cruz Biotechnology, 1:2000 dilution). Results are shown in FIG. 2. All procedures were performed with standard molecular biology protocols for protein expression studies.

RNA was processed for real-time PCR. FIG. 3 shows the normalized ratio of Ppargc1α mRNA to beta-actin mRNA detected using a rt-PCR assay at various concentrations of TQS-168 (0.7 to 20 μM).

### Conclusion

FIGS. 2 and 3 respectively demonstrate that TQS-168 induces PGC-1α protein expression and Ppargc1α mRNA expression in murine BV2 cells in vitro at concentrations ranging from 0.7 μM to 20 μM.

### 3.1.3. Example 3—TQS-168 Inhibits Inflammatory Cytokine Secretion from LPS-Stimulated BV2 Cells In Vitro at Concentrations Ranging from 1 μM to 20 μM (CBA Assay)

Lipopolysaccharide (LPS) is a natural ligand of the TLR4/CD14 complex, which is highly expressed on myeloid cells.

LPS was used to induce cytokine secretion from BV-2 cells. Cells were incubated with TQS-168 at various concentrations to assess whether TQS-168 could suppress the LPS-promoted release of various cytokines from in vitro cell cultures. Cytokine secretion was measured by cytometric bead array (CBA) fluorescence-activated cell sorting (FACS).

Briefly, a vial of BV2 frozen stock (1 million cells per ml in complete medium) was thawed into 10 mL of complete medium per vial for a total of 4 vials. The vials were then centrifuged at 1800 rpm for 3 minutes to wash away freezing media. The four vials were then pooled into 25 mL of complete medium. TQS-168 was prepared in DMSO.

Cells were incubated for 24 hours in the presence of medium (negative control), LPS (positive control), DMSO+LPS (positive control, controlling additionally for presence of DMSO in the TQS-168 stock solution), or LPS+TQS-168 at final concentrations of 1 μM, 5 μM, 10 μM, and 20 μM.

After the well plates were incubated overnight for 24 hours, they were centrifuged at 1800 rpm for 5 minutes. Next, 150 μl from each well was transferred to a new set of plates, of which 50 μl was used for CBA. The plates were then centrifuged with the cells, followed by addition of 100 μl of DAPI (50 ml of PBS+10 μl of DAPI stock at 1:5000 dilution). The plates were then incubated in the dark for 5 minutes, followed by addition of 100 μl of PBS and centrifuged at 1800 rpm for 5 minutes. Finally, the plates were resuspended in 200 μl of PBS and tested.

### CBA Protocol

Multiplexed cytometric bead array (CBA) assay was performed according to standard techniques to detect presence in the cell culture medium of secreted murine TNFα, IL-6, IFNγ, IL-12, monocyte chemoattractant protein 1 (MCP-1), and IL-10.

### Conclusion

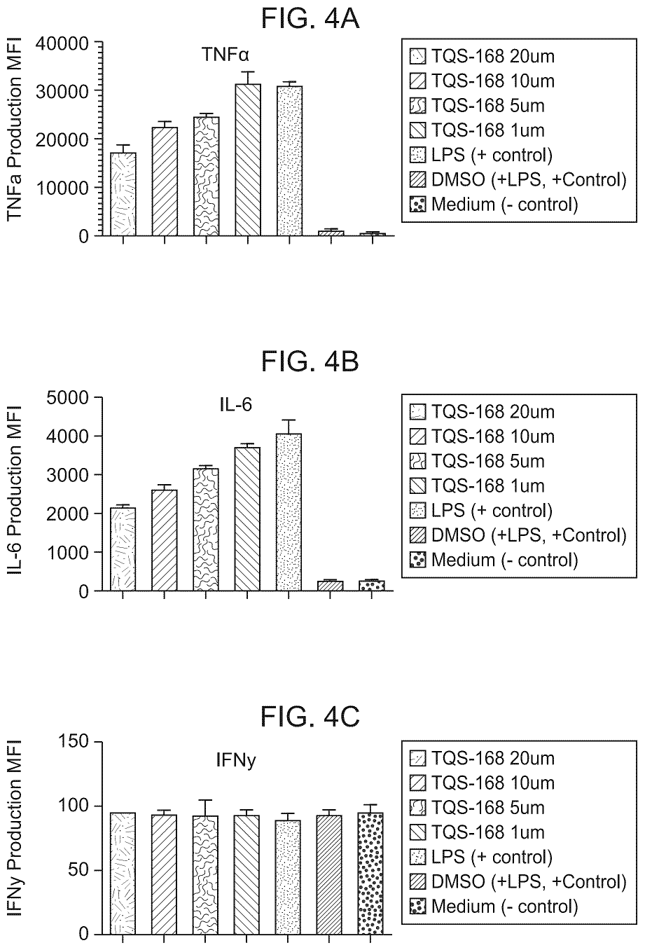

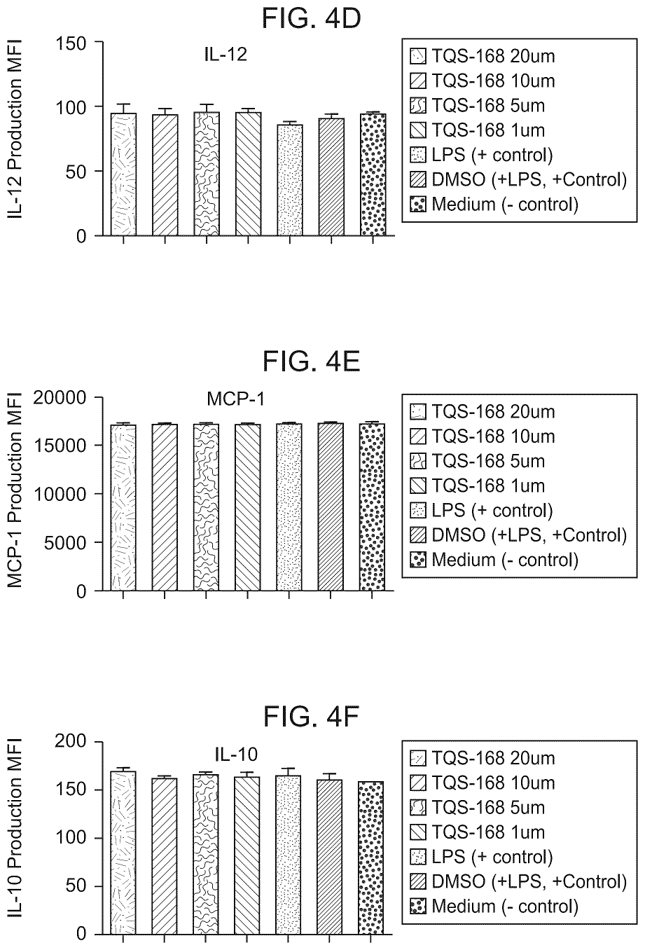

As shown in FIGS. 4a-4f, BV2 cells treated with 100 ng/ml LPS displayed strong release of TNFα and IL-6 pro-inflammatory cytokines, while secretion of INF-γ, IL-10, IL-12 and MCP-1 were not significantly induced. TQS-168 was able to reduce the LPS-induced secretion of TNFα and IL-6 from BV2 cells at concentrations ranging from 1 μM-20 μM. Reductions of both TNFα (FIG. 4a) and IL-6 (FIG. 4b) by TQS-168 were concentration-dependent.

### 3.1.4. Example 4—TQS-168 Inhibits LPS-Induced TNFα Secretion from BV2 Myeloid Cells In Vitro at 5 μM and 20 μM (CBA Assay)

A total of 4-11 replicates of BV2 cultures in different conditions were investigated. Frozen BV2 microglia cell line was thawed and propagated in complete media (RPMI, 10% heat inactivated FBS, 1% L-glutamine, 1% Pen-Strep) until growth rate reached log phase.

Cells were stimulated with 100 ng/ml of LPS for 24 hours. DMSO (1:1000 final dilution) or TQS-168 in DMSO (1:1000 final dilution) at either 5 μM or 20 μM were added to cell cultures. After 24 hours stimulation, supernatant was collected for cytokine analysis with CBA assays (BD Biosciences) per manufacturer's protocols. TNFα express was normalized to DMSO treated condition.

ANOVA was used for statistical analysis with significant threshold at p-value<0.05.

### Conclusion

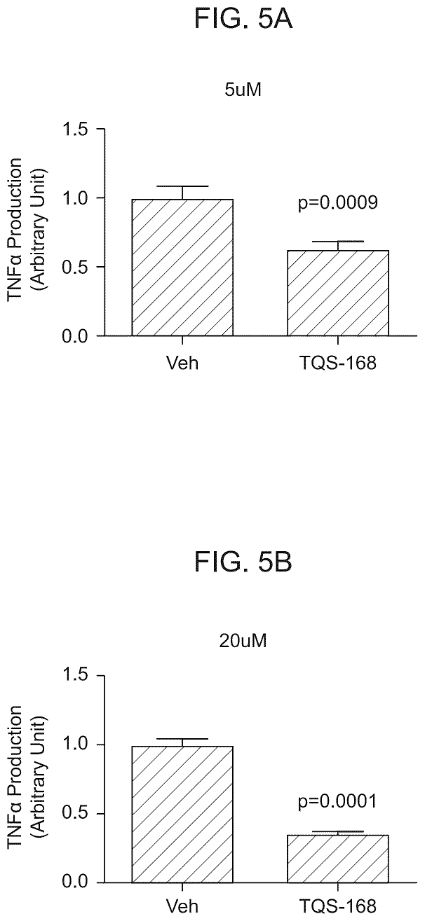

As shown in FIGS. 5a and 5b, TQS-168 at 5 μM and 20 μM, respectively, can suppress TNFα production by LPS-stimulated BV-2 myeloid cells, as compared to control.

### 3.1.5. Example 5—TQS-168 Inhibits TNFα Release by BV2 Cells Stimulated with LPS In Vitro at Concentrations of 0.3 μM to 10 μM (ELISA)

### TNFα ELISA Procedure

First, 100 μl of supernatant was removed from Cell Plate and transferred to a Dilution Plate, which was then centrifuged at 216×g for 10 minutes to remove particulates. The dilution plate was either assayed immediately or aliquots were taken and stored at stored at ≤−20° C.; repeated freeze-thaw cycles were avoided.

Next, a standard curve was prepared by first pipetting 900 ul of Calibrator Diluent RD5K into the 700 pg/mL tube, followed by 200 μl of the appropriate calibrator diluent in the remaining tubes. The stock solution was used to produce a dilution. The resulting tubes were then thoroughly mixed. The Mouse TNFα Standard (700 pg/mL) served as the high standard, and the Calibrator Diluent RD5T served as the zero standard at 0 pg/ml.

Assay Diluent RD1-63 (50 ul) was then added to the center of each well and mixed before and during its use. Then, 50 ul of either standard, control, or sample was added to the center of each well, and covered with adhesive strip. The plate was then mixed for 1 minute and incubated for 2 hours at room temperature.

Each well was then aspirated and washed by filling each well with Wash Buffer (400 ul) five times. After the last wash, the remaining Wash Buffer was removed by aspirating or decanting. TNFα IL-6 conjugate (100 ul) was then added to each well, covered with adhesive strip, incubated for 2 hours at room temperature, and washed and/or aspirated five times.

Substrate solution (100 ul) was then added to each well, incubated in the dark for 30 minutes at room temperature, followed by addition of 100 μl of Stop Solution and mixed. The optical density of each well was determined within 30 minutes by using a microplate reader set to 450 nm.

### Measurement Settings and Parameters and Data Processing

Plates were read on a Spectrostar Nano machine with built-in MARS data analysis at 450 nM and 570 nM.

### Optimization Parameters

Titration of LPS stimulation was performed to optimize the assay dynamic range.

For TNFα ELISA, the BV-2 cells were very responsive to low concentrations of LPS. A concentration range of 0.1 ng/ml to 1,000 ng/ml was tested. 0.3 ng/ml LPS produced enough TNFα release (8-10 fold above background) from these cells after 22 hours stimulation without saturating the linear range of the ELISA detection system. If higher LPS concentrations are used for stimulating BV-2 cells, a sample solution is advised to stay within the linear range of the detection system. The current TNFα protocol uses 10,000 cells per well in 96 well plate. A cell count titration can optimize S/B ratio. Miniaturization from 96 well to 384 well is also feasible with this assay.

### Conclusion

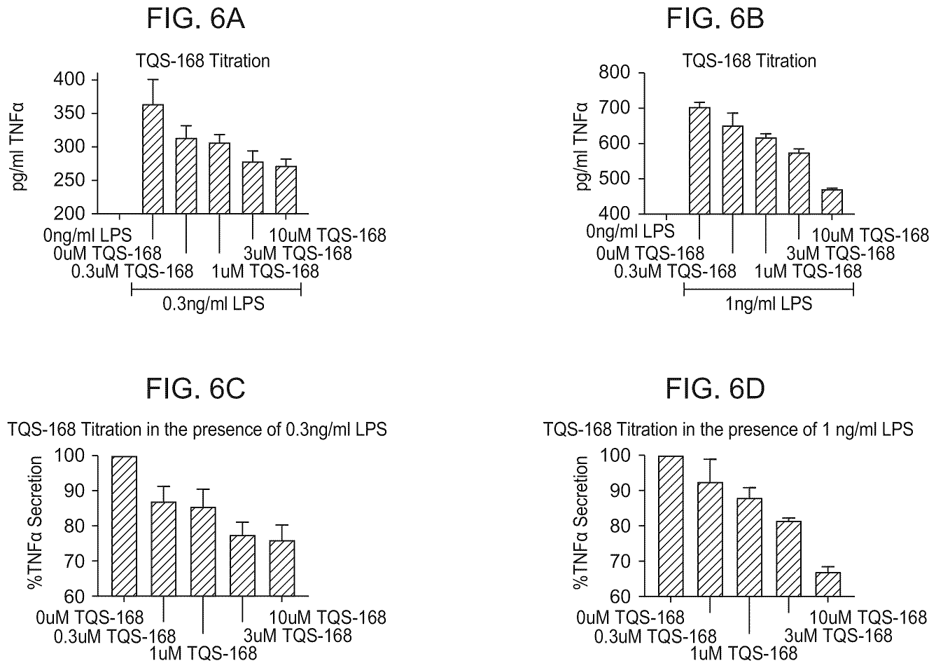

FIGS. 6a-6d show the dose response of TQS-168-mediated decrease of pro-inflammatory cytokine TNFα release from microglia BV2 cells using ELISA at 24 hours. FIG. 6a and FIG. 6c show the absolute (FIG. 6a) and relative (FIG. 6b) inhibition of TNFα secretion by BV2 cells stimulated with 0.3 ng/ml of LPS. FIG. 6b and FIG. 6d show the absolute (FIG. 6b) and relative (FIG. 6d) inhibition of TNFα secretion by BV2 cells stimulated with 1 ng/ml of LPS.

In BV-2 cells treated with 0.3 ng/ml of LPS, administration of TQS-168 suppressed TNFα production in a concentration-dependent manner, with up to about 25% suppression observed for cells administered with 10 μM of TQS-168.

Similarly, in BV-2 cells treated with 1 ng/ml of LPS, administration of TQS-168 suppressed TNFα production in a concentration-dependent manner, with up to about 35% inhibition for cells administered with 10 μM of TQS-168.

### 3.1.6. Example 6—TQS-168 Inhibits LPS-Induced TNF-α Production of Primary Human Myeloid Cells

### Study Methodology

Peripheral blood mononuclear cells (PBMC) from 4 different healthy volunteers were used in this study. Fresh blood samples were collected at Stanford Blood Center and processed for PBMC isolation with Ficoll gradient. PBMC samples were stored in liquid nitrogen at −80° C. for subsequent analysis of TNF-α production.

For TNF-α stimulation, frozen PBMC samples were thawed and rested at 37° C. before cells were stimulated with 100 ng/ml of LPS for 24 hours. DMSO (1:1000 final dilution) or TQS-168 in DMSO (1:1000 final dilution) at various concentrations was added to cell cultures of LPS-stimulated PBMC to evaluate the effects of T-168 on TNF-α production of human primary myeloid cells. After 24 hours stimulation, supernatant samples from various conditions were collected and TNF-α concentrations in the supernatants analyzed with cytometric bead array (CBA) assay per protocols from BD Biosciences. TNF-α was quantified by median fluorescent intensity reading (MFI). Samples were analyzed directly after staining with LSRII flow cytometer.

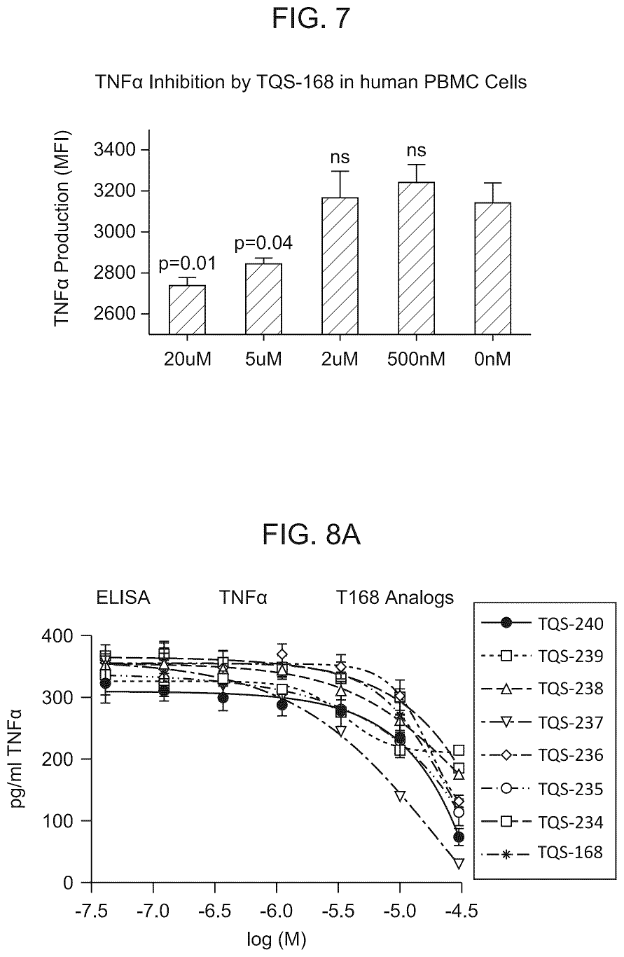

As shown in FIG. 7, in human PMBC cells treated with 100 ng/ml LPS, administration of TQS-168 at 5 μM and 20 μM led to statistically significant suppression of TNFα production as compared to no treatment.

### 3.1.7. Example 7—TQS-168 Analogs Inhibit TNF-α and IL-6 Secretion from LPS-Stimulated Mouse Myeloid BV2 Cells

The ability of TQS-168 analogs TQS-240, TQS-239, TQS-238, TQS-237, TQS-236, TQS-235, and TQS-234 to inhibit LPS-stimulated release of TNFα and IL-6 was assessed. Structures are shown in Table 1 below.

TABLE 1
TQS-168 and analog structures

| | |
|---|---|
| 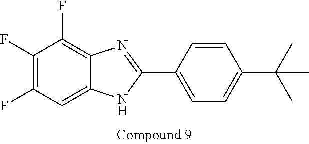 [SMILES: CC(C)(C)c1ccc(-c2nc3c(F)c(F)c(F)cc3[nH]2)cc1] | TQS-240 |
| 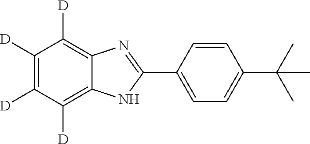 [SMILES: [2H]c1c([2H])c([2H])c2[nH]c(-c3ccc(C(C)(C)C)cc3)nc2c1[2H]] | TQS-239 |
| 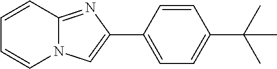 [SMILES: CC(C)(C)c1ccc(-c2cn3ccccc3n2)cc1] | TQS-238 |
| 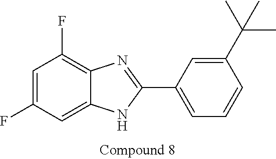 [SMILES: CC(C)(C)c1cccc(-c2nc3c(F)cc(F)cc3[nH]2)c1] | TQS-237 |
| 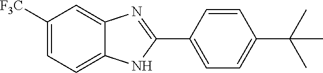 [SMILES: CC(C)(C)c1ccc(-c2nc3cc(C(F)(F)F)ccc3[nH]2)cc1] | TQS-236 |
| 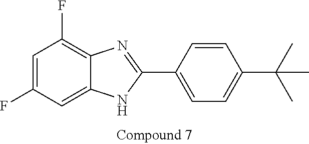 [SMILES: CC(C)(C)c1ccc(-c2nc3c(F)cc(F)cc3[nH]2)cc1] | TQS-235 |
| 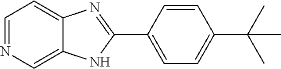 [SMILES: CC(C)(C)c1ccc(-c2nc3ccncc3[nH]2)cc1] | TQS-234 |

The TNFα ELISA assay is described in Example 5. The IL-6 assay is described below.

### IL-6 ELISA Procedure

[1] First, 100 μl of supernatant from cell plate was removed and transferred to a dilution plate, followed by centrifugation of the dilution plate at 216×g for 10 minutes to remove particulates. The dilution plate was then either assayed immediately or aliquots were taken and stored at ≤−20° C.; repeated freeze-thaw cycles should be avoided.
[2] Mouse IL-6 Standard (500 pg/mL) served as the high standard, and the Calibrator Diluent RD5T served as the zero standard at 0 pg/ml.
[3] Assay Diluent RD1-14 (50 μl) was then added to the center of each well and mixed before and during its use. Then, 50 μl of standard, control, or sample was added to the center of each well, and covered with adhesive strip. The plate was then mixed for 1 minute and incubated for 2 hours at room temperature.
[4] Each well was then aspirated and washed by filling each well with wash buffer (400 ul) five times. After the last wash, the remaining Wash Buffer was removed by aspirating or decanting. Mouse IL-6 conjugate (100 ul) was then added to each well, covered with adhesive strip, incubated for 2 hours at room temperature, and washed and/or aspirated five times.

Substrate solution (100 ul) was then added to each well, incubated in the dark for 30 minutes at room temperature, followed by addition of 100 ul of Stop Solution and mixed. The optical density of each well was determined within 30 minutes by using a microplate reader set to 450 nm.

### Results

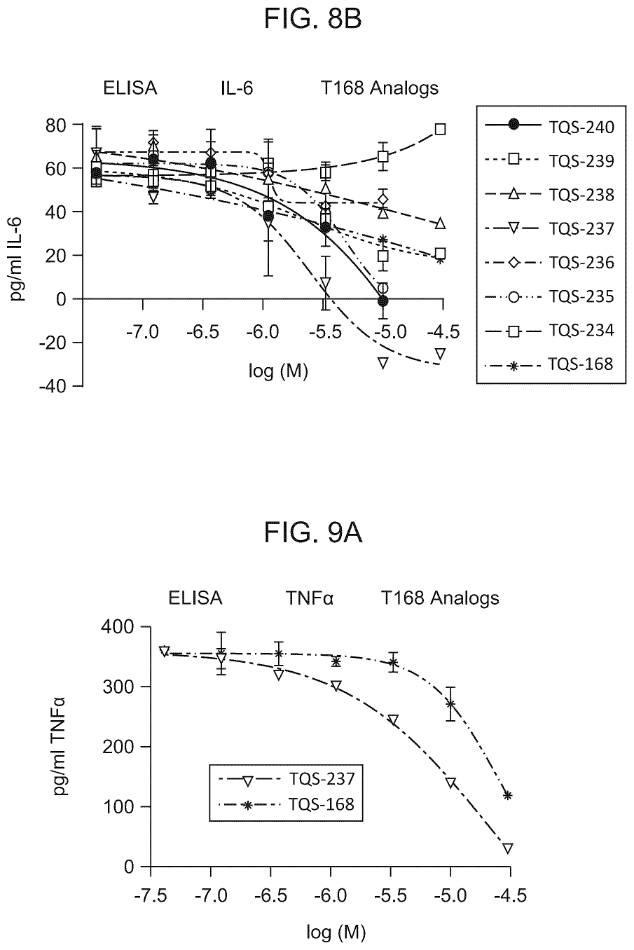

BV-2 cells treated with TQS-168 analogs TQS-240, TQS-239, TQS-238, TQS-237, TQS-236, TQS-235, and TQS-234 showed decrease in LPS-stimulated TNFα secretion and IL-6 secretion. FIG. 8A shows the concentration-response of TQS-168 analogs in the TNFα assay. FIG. 8B shows the concentration-response of TQS-168 analogs in the IL-6 assay.

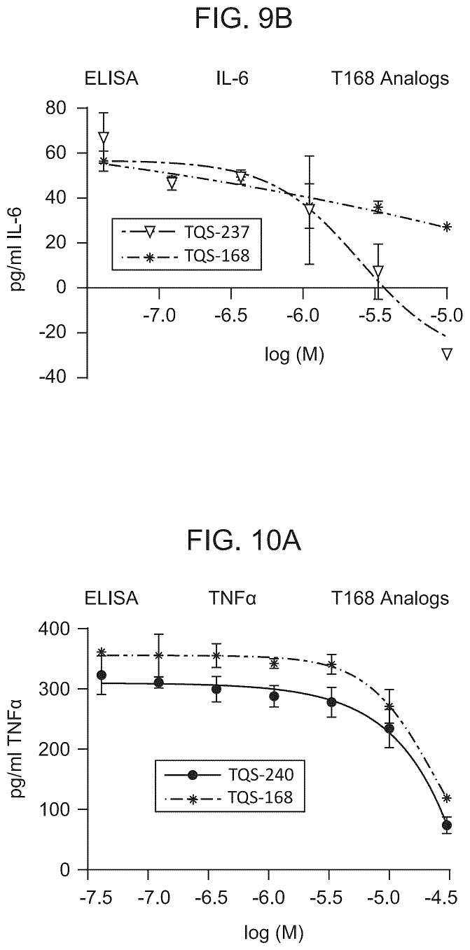

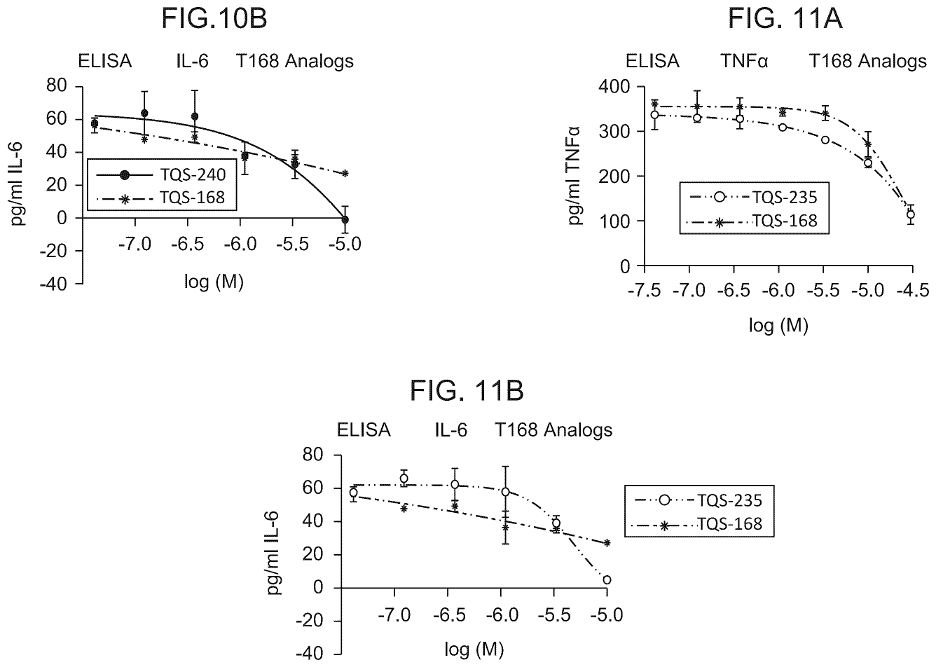

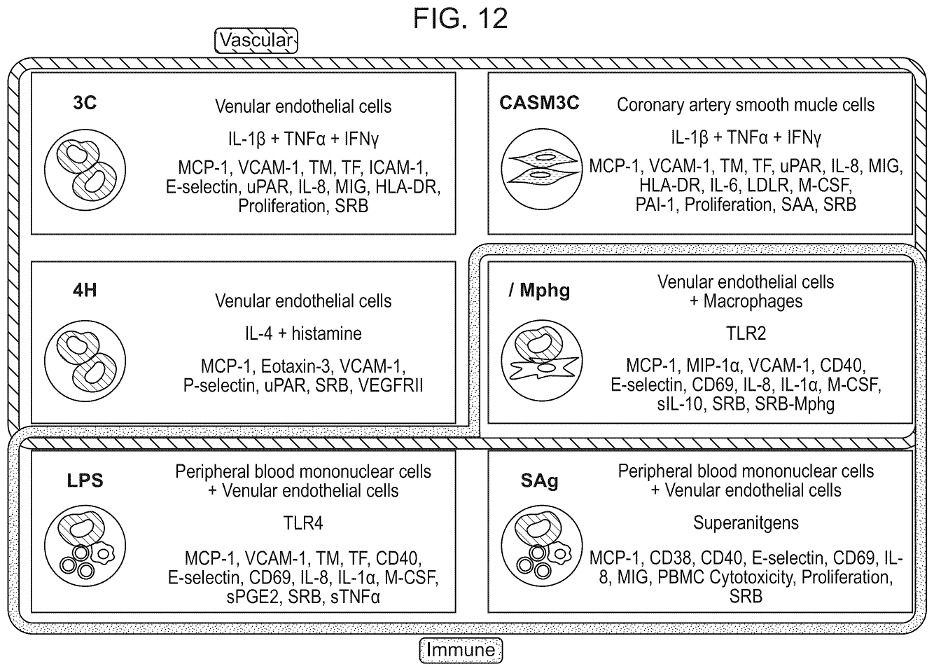

FIG. 9 provides a subset of the data, providing a direct comparison of the concentration-response curve of TQS-237 and TQS-168 in the TNFα (FIG. 9A) and IL-6 (FIG. 9B) assays. FIG. 10 provides a subset of the data, presenting a direct comparison of the concentration-response curve of TQS-240 and TQS-168 in the TNFα (FIG. 10A) and IL-6 (FIG. 10B) assays. FIG. 11 provides a direct comparison between TQS-235 and TQS-168 in the TNFα (FIG. 11A) and IL-6 assays (FIG. 11B).

### 3.1.8. Example 8—Human Phenotypic Profiling of TQS-168

The goal of this study was to survey the effects of TQS-168 across a broad array of human cell systems.

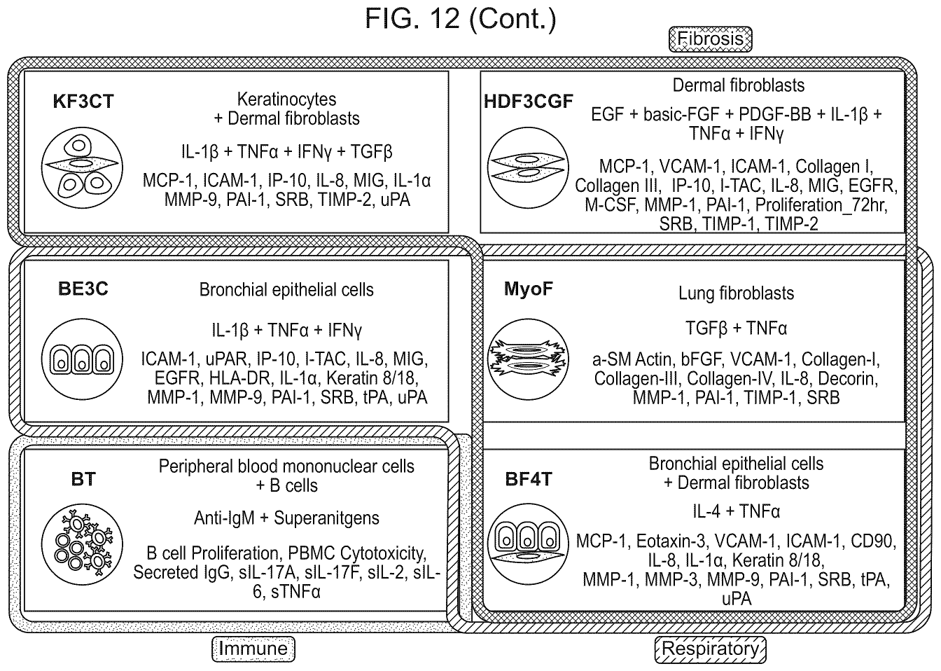

TQS-168 was characterized in the Eurofins BioMAP Diversity PLUS panel of 12 human primary cell-based systems (FIG. 12). These systems are designed to model complex human tissue and disease biology of the vasculature, skin (fibrosis), pulmonary and immune systems and provide test agent characterization in an unbiased way across a broad set of systems modeling various human disease states. Quantitative measurements of biomarker activities across this broad panel, along with comparative analysis of the biological activities of known bioactive agents in the BioMAP reference database were used to predict the safety, efficacy and function of TQS-168.

### Results: BioMAP Profiles in Diversity PLUS Panel for TOS-168

A BioMAP profile in the Diversity PLUS Panel was performed for TQS-168 at concentrations of 20 μM, 6.7 μM, 2.2 μM, and 740 nM.

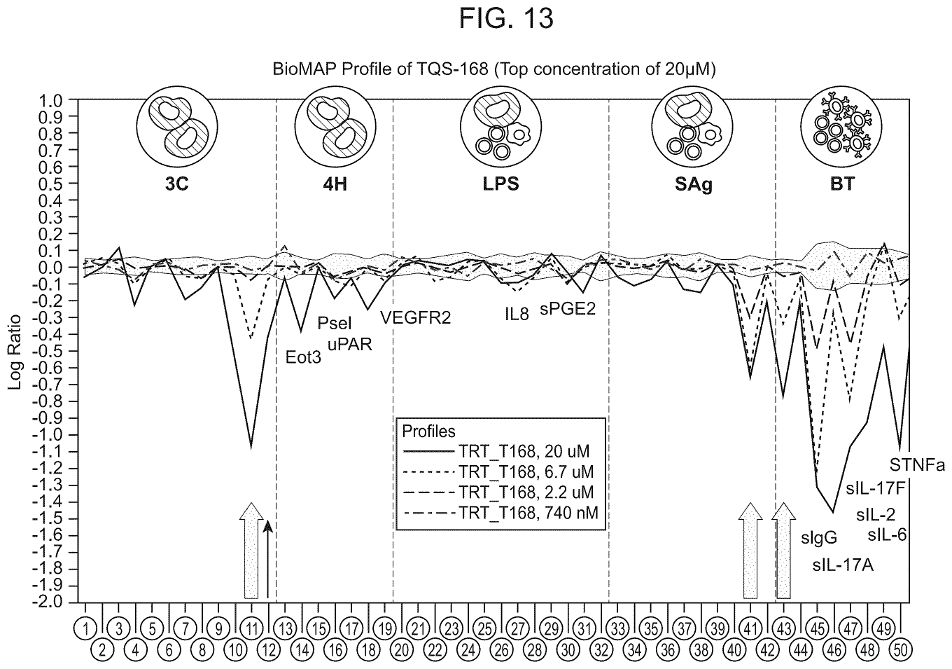

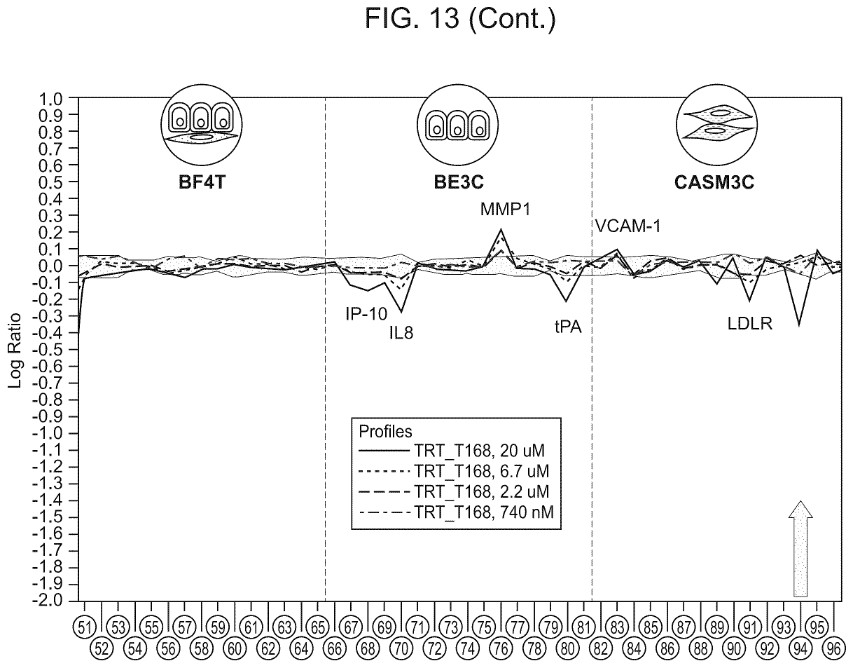

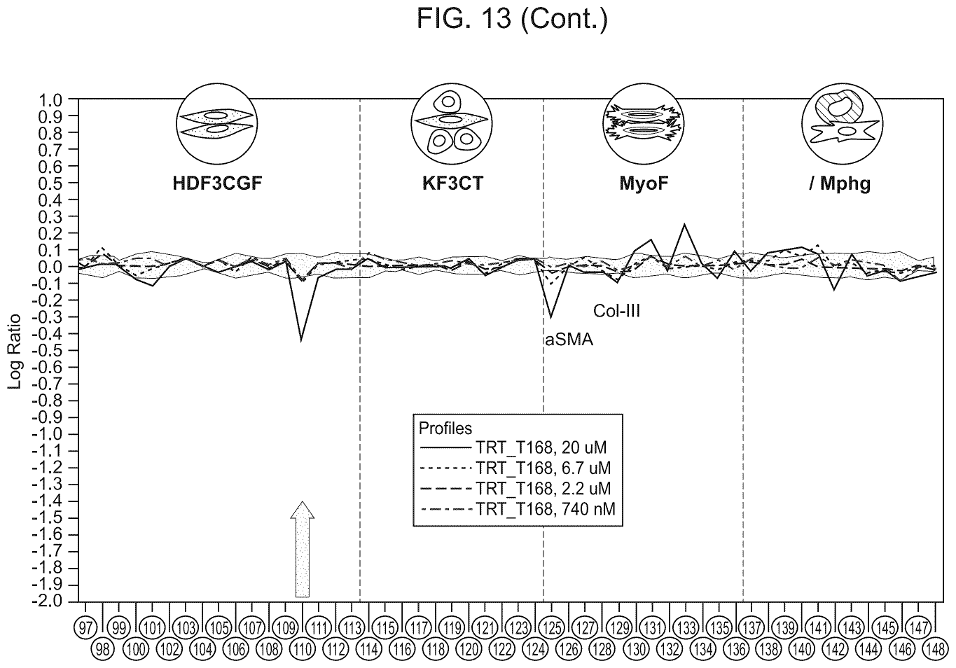

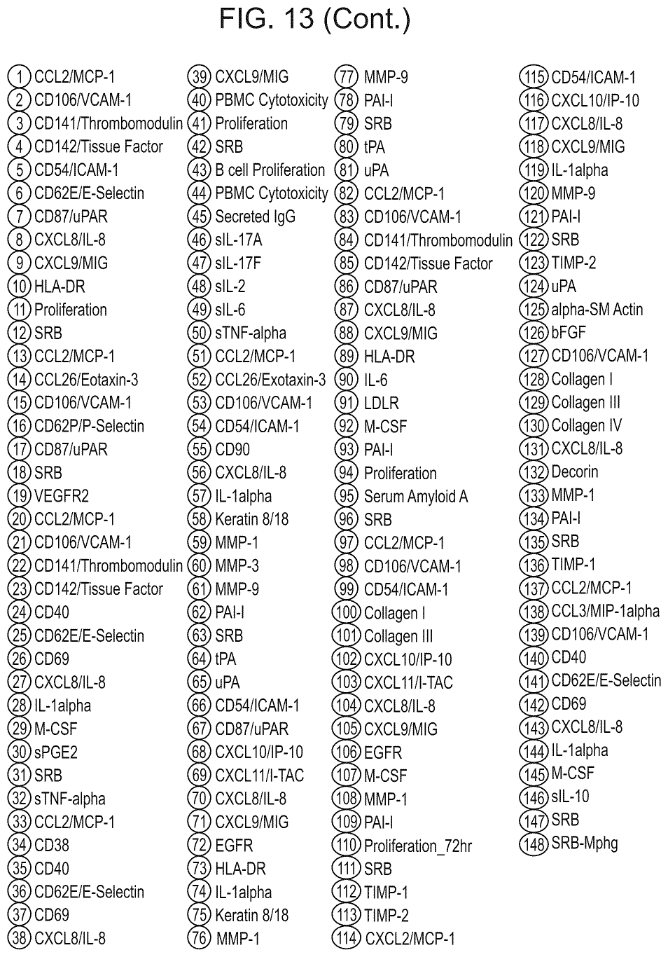

Results are summarized in FIG. 13. The Y-axis represents a log-transformed ratio of the biomarker readouts for the test agent-treated sample (n=1) over vehicle controls (average of ≥6 vehicle controls from the same plate). The grey region around the Y-axis represents the 95% significance envelope that was generated from the upper and lower bound values of historical vehicle controls. Biomarker activities were annotated when two or more consecutive concentrations change in the same direction relative to vehicle controls, were outside of the significance envelope, and have at least one concentration with an effect size >20% (|log₁₀ ratio|>0.1). Biomarker key activities were described as modulated if these activities increase in some systems, but decreased in others. Concentrations that demonstrated cytotoxicity were removed.

The results summarized in FIG. 13 show that TQS-168 was active across most systems, but was cytotoxic to endothelial cells (black arrow) at 20 μM. The BioMAP profile also showed that TQS-168
(i) was anti-proliferative to endothelial, B cells, T cells, coronary artery SMC & fibroblasts (grey arrows in FIG. 13);
(ii) decreased a variety of immune markers, notably sIgG, sIL-17A, sIL-17F, sIL-2, sTNFα, and sIL-6 (from B and T cells, not monocytes);
(iii) decreased inflammatory markers, specifically Eotaxin-3, P-selectin, IL-8, PGE2, IP-10;
(iv) increased VCAM-1; and
(v) modulated tissue remodeling markers: uPAR, MMP1, tPA, alpha-SMA, Collagen III.

The highest concentration of TQS-168, 20 μM, showed the greatest decrease in inflammatory markers sIgG, sIL-17A, sIL-17F, sIL-2, sTNFα, and sIL-6 from B and T cells.

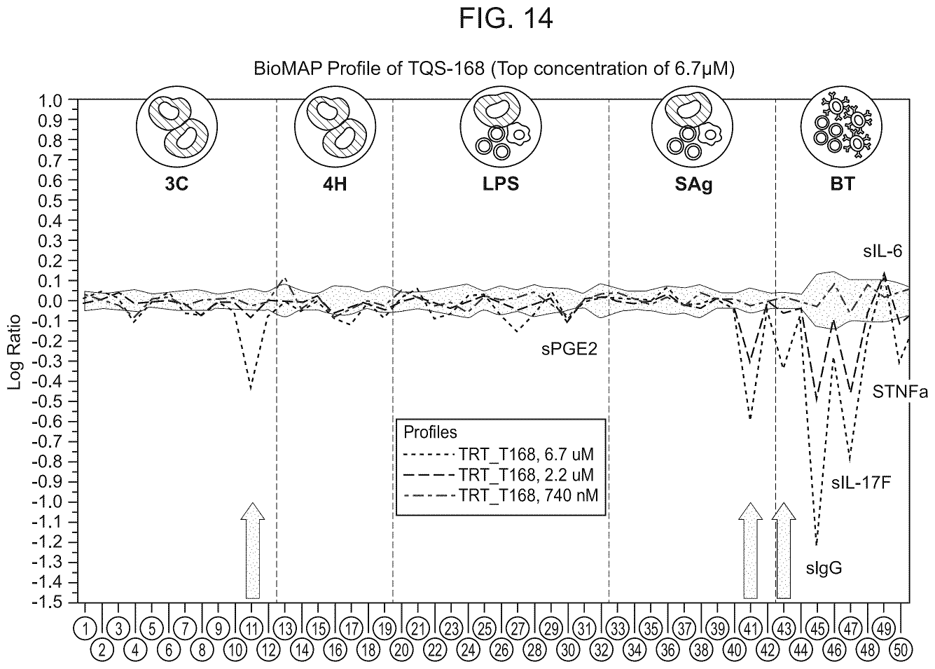

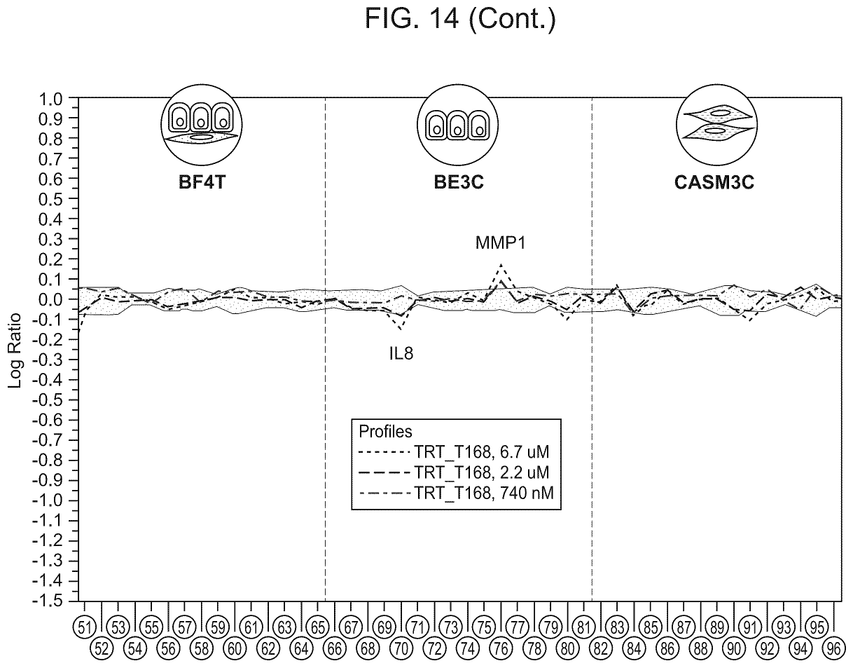

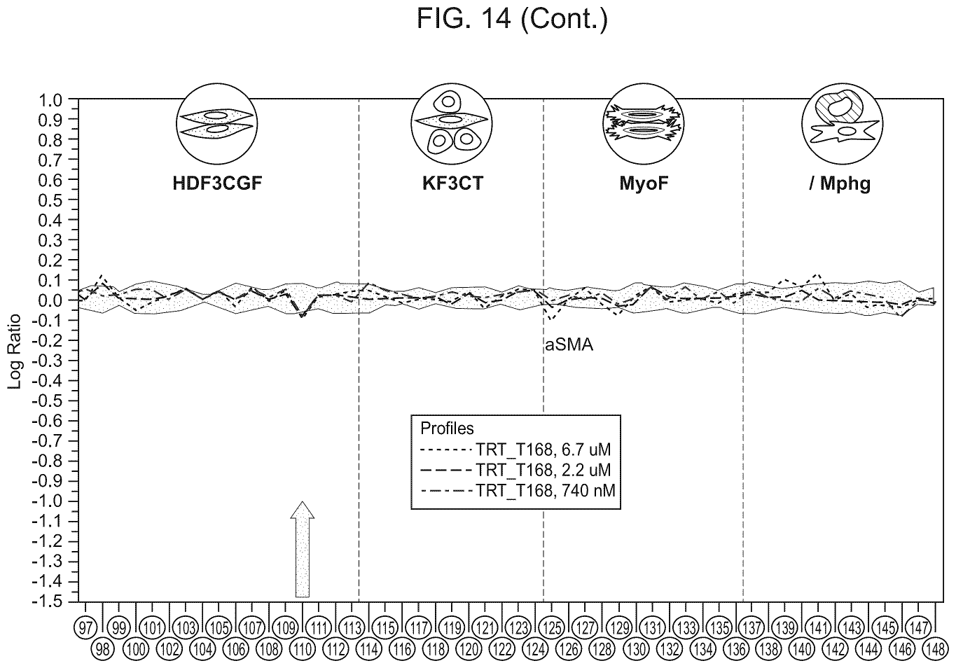

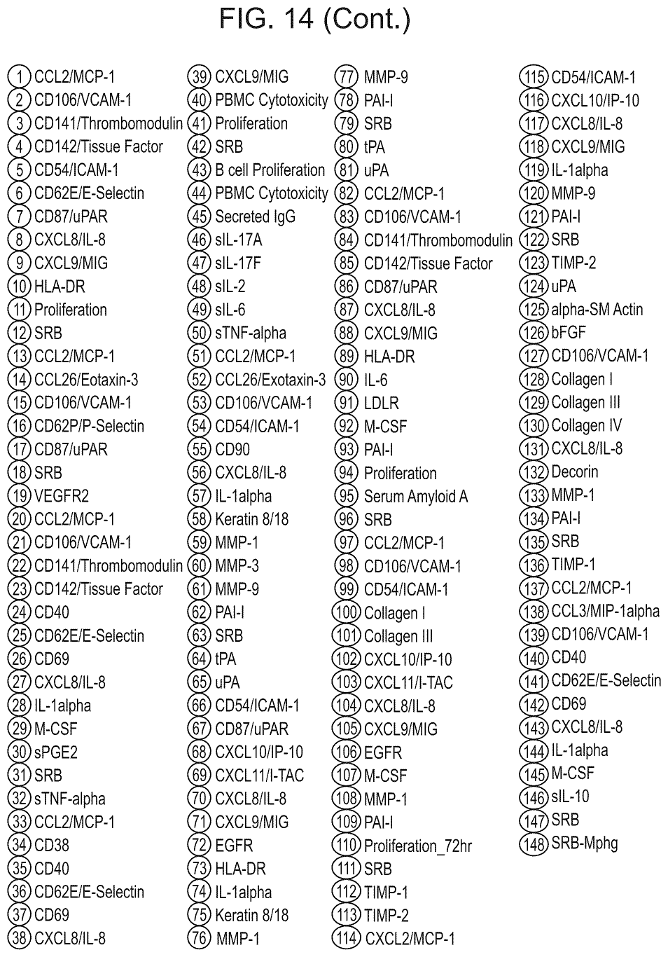

A BioMAP profile was also performed for TQS-168 at concentrations of 6.7 μM, 2.2 μM, and 740 nM. These results are shown in FIG. 14, which for clarity omits results obtained at 20 μM. The results show that TQS-168
(i) at varying concentrations was active at non-cytotoxic concentrations;
(ii) was anti-proliferative to endothelial cells, T cells, B cells & fibroblasts (grey arrows);
(iii) decreased inflammatory markers: sPGE2 (from monocytes), IL-8;
(iv) decreased immune markers: sIgG, sIL-17-F, sTNFα (from B and T cells, not monocytes);
(v) increased sIL-6; and
(vi) modulated tissue remodeling markers: MMP1, alpha-SMA.

The highest concentration of TQS-168, at 6.7 μM, showed the greatest decrease in inflammatory markers sIgG, sIL-17F, and sTNFα from B and T cells. The BioMAP report indicated that TRT_T168 impacts inflammation-related activities (decreased sTNFα, IL-8, sPGE2), immunomodulatory activities (decreased sIgG, sIL-17F; increased sIL-6), and tissue remodeling activities (decreased αSMA; increased MMP-1).

An unsupervised search of the BioMAP Reference Database of >4,500 agents with TQS-168 at 20 μM was performed. Overlay of the top similarity match from common biomarker readouts were annotated when the readout for both profiles was outside of the significance envelope with an effect size >20% (|log₁₀ ratio|>0.1) in the same direction. Similarity search results were filtered and ranked. Profiles were identified as having mechanistically relevant similarity if the Pearson's correlation coefficient is ≥0.7. Additional information is found Methods documentation from Eurofins. Systems with overt cytotoxicity are not annotated. Concentrations with more than three systems with cytotoxicity were considered overtly cytotoxic and were not included in the Similarity Search Analysis. In an unsupervised search for mathematically similar compound profiles from the BioMAP Reference Database, TRT_T168 at the top concentration tested (20 μM), which was cytotoxic to endothelial cells in the BioMAP 3C system, was most similar to cerivastatin (1.1 μM) (Pearson's correlation coefficient, r=0.873). The Pearson's correlation coefficient between these two profiles was above the determined threshold of r=0.7 indicating these compounds shared mechanistically relevant similarity.

The top database similarity search result for TQS-168 (20 μM) was Cerivastatin (1.1 μM). Cerivastatin is an HMG-COA reductase inhibitor. There were 19 common activities that are annotated within the following systems: 4H (SRB), LPS (SRB), Sag (CD69, Prolif, SRB), BT (Prolif, Pcyto, sIgG, sIL-17A, sIL-17F, sIL-2, sIL-6, sTNFα), CASM3C (Prolif), HDF3CGF (Prolif 72), MyoF (αSMA, Collagen III, IL-8, MMP-1). Please note, systems with detectable cytotoxicity are excluded from annotation (3C).

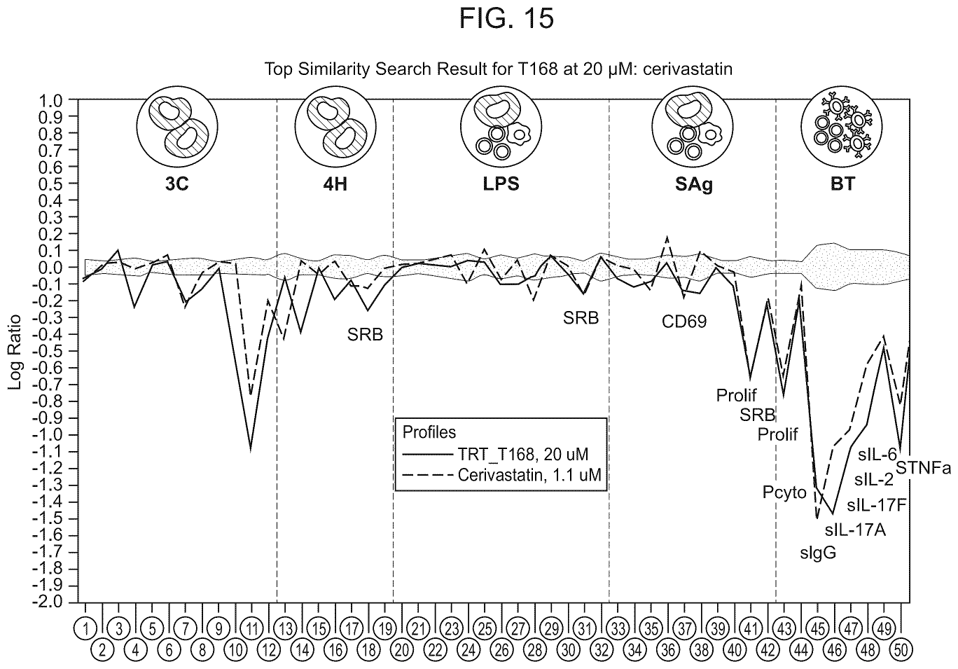

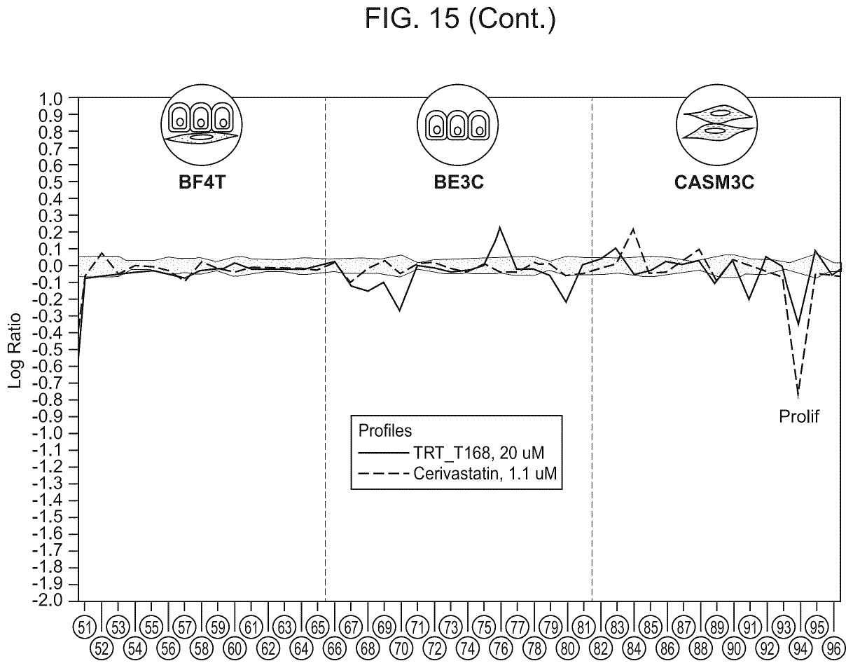

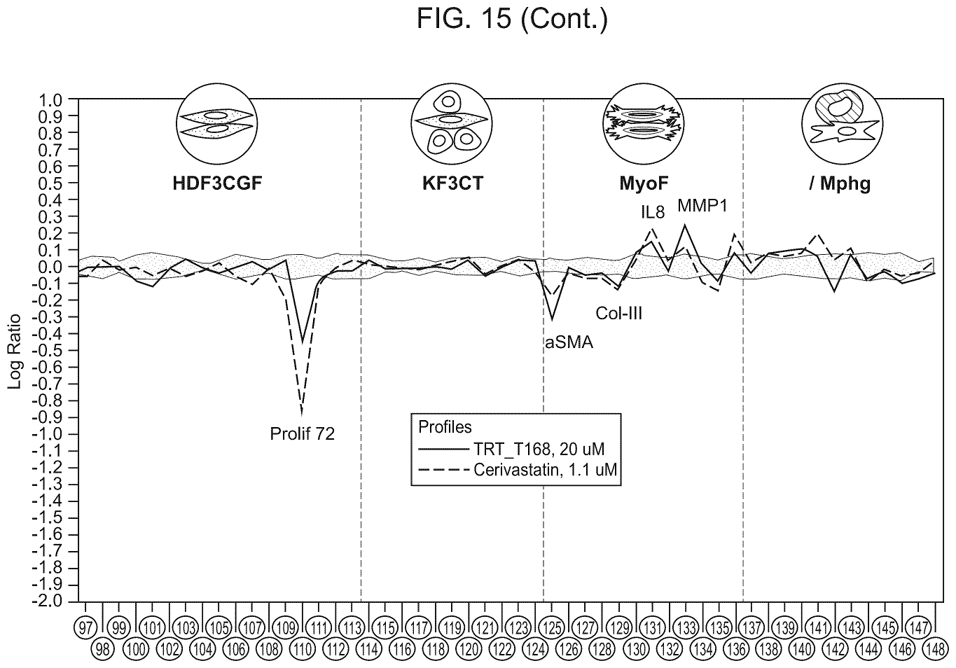

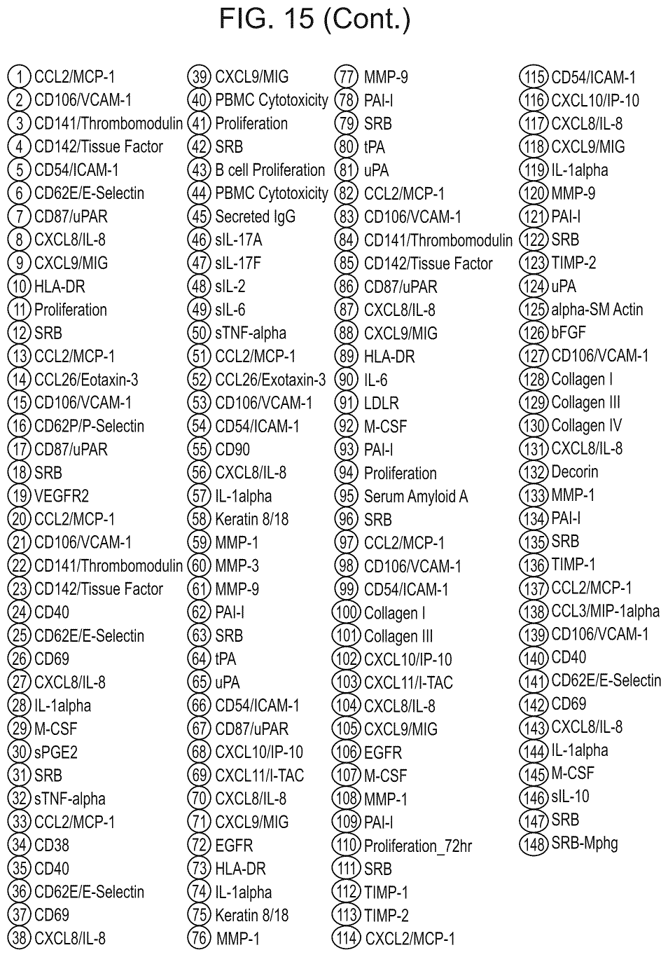

FIG. 15 overlays profiles of TQS-168 at 20 μM and cerivastatin at 1.1 μM.

In an unsupervised search for mathematically similar compound profiles from the BioMAP Reference Database, the profile for TQS-168 at 6.7 μM, the highest nontoxic concentration tested, was most similar to deferoxamine mesylate (13 μM) (Pearson's correlation coefficient, r=0.880). Deferoxamine (also known as desferroxamine, desferrioxamine, desferroxamine) is a chelator of iron and aluminum (used clinically) and has been shown to delay disease onset and extend lifespan in a transgenic mouse model of ALS (Lee, 2015; PMID: 26002422). Deferoxamine increases HIF-1alpha (iron metabolism regulates the HIF-1alpha pathway; the HIF-1 pathway is dysregulated in ALS (PMID: 20977930)).

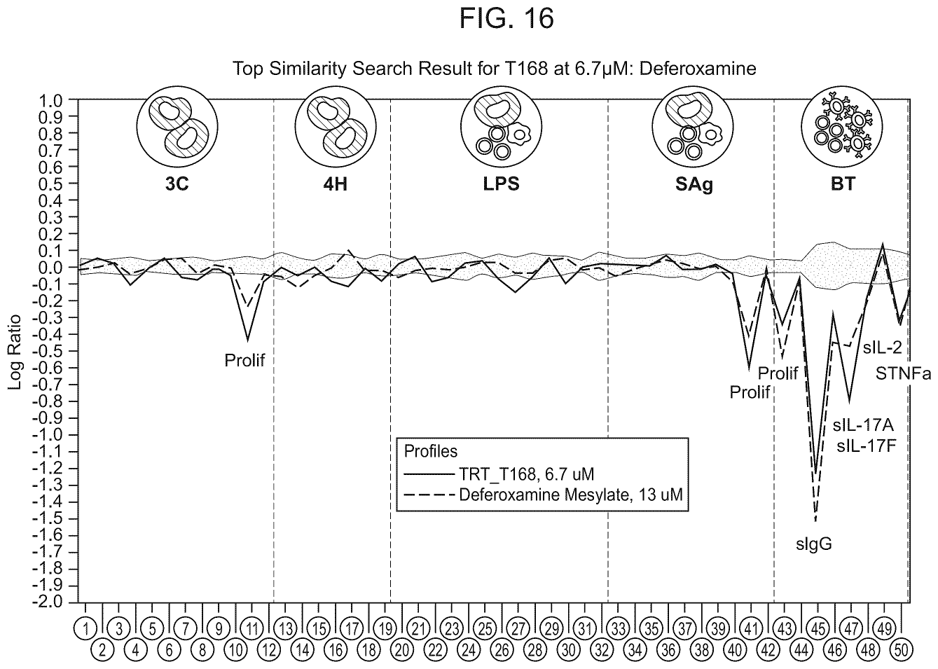

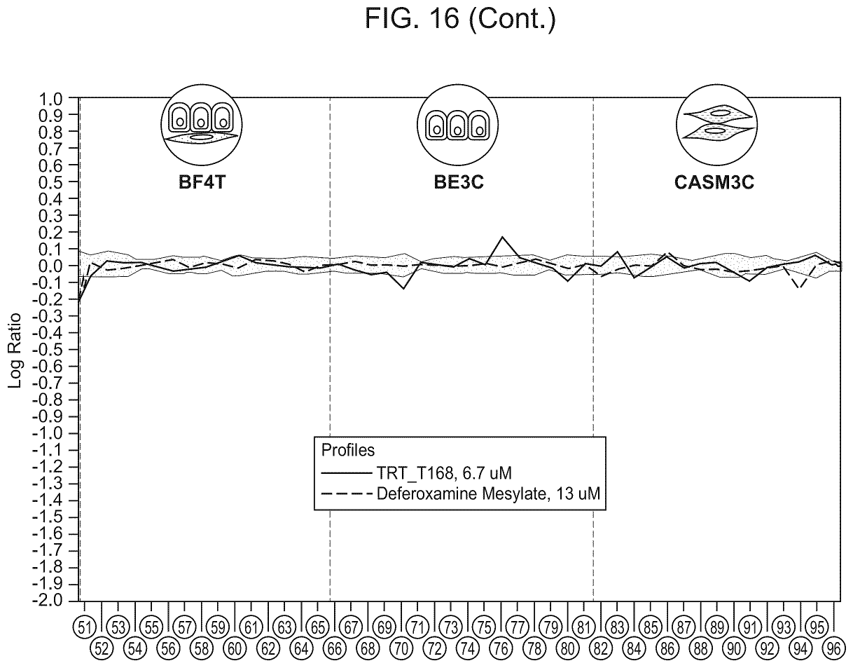

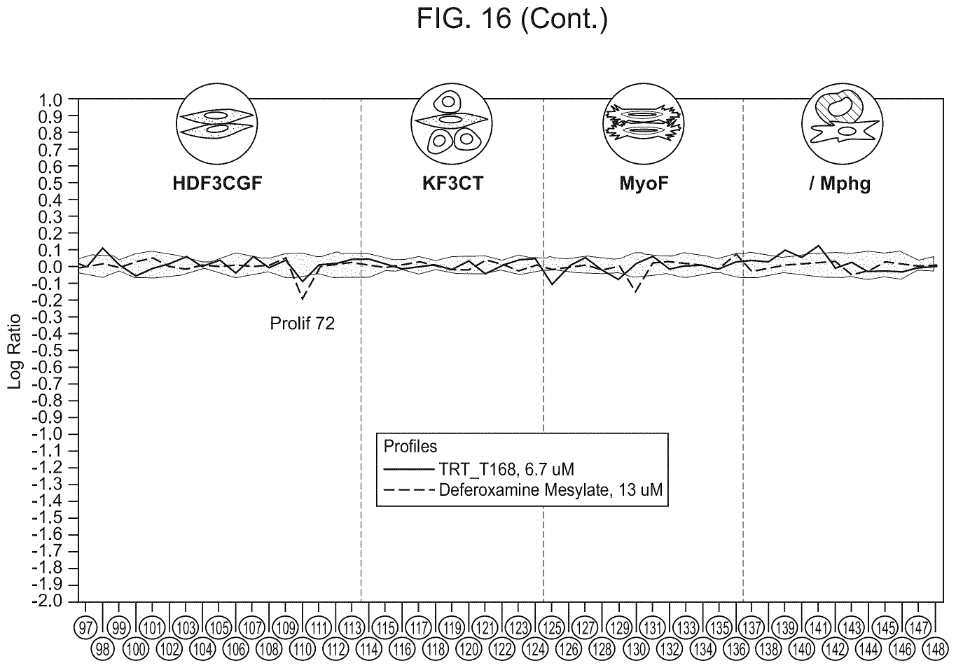

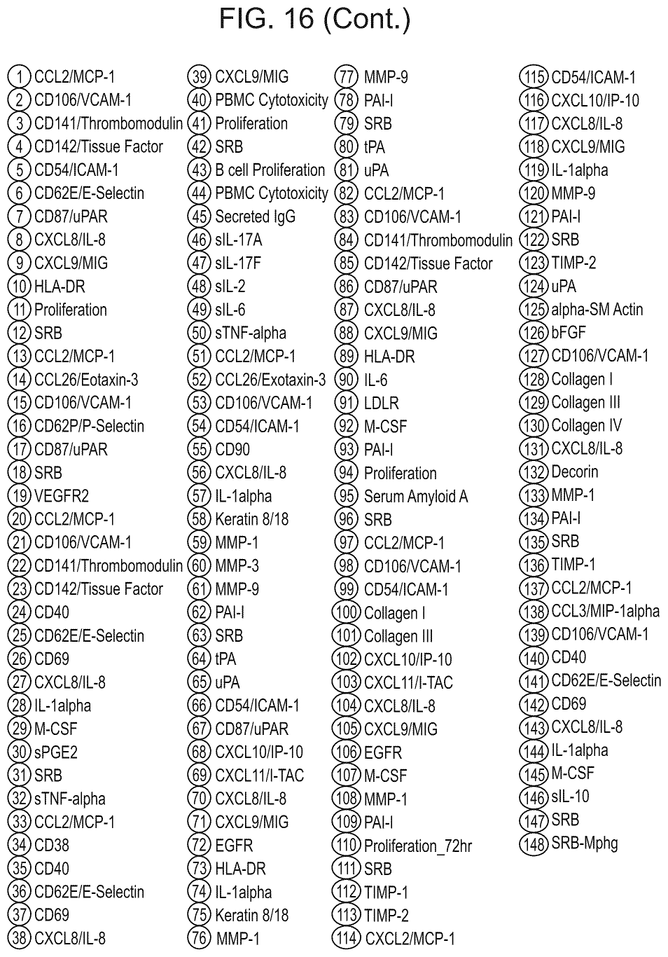

The comparison is shown in FIG. 16. TQS-168 at a concentration of 6.7 μM showed nine common activities with deferoxamine that are annotated within the following systems: 3C (Prolif), Sag (Prolif), BT (Prolif, sIgG, sIL-17A, sIL-17F, sIL-2, sTNFα), and HDF3CGF (Prolif 72).

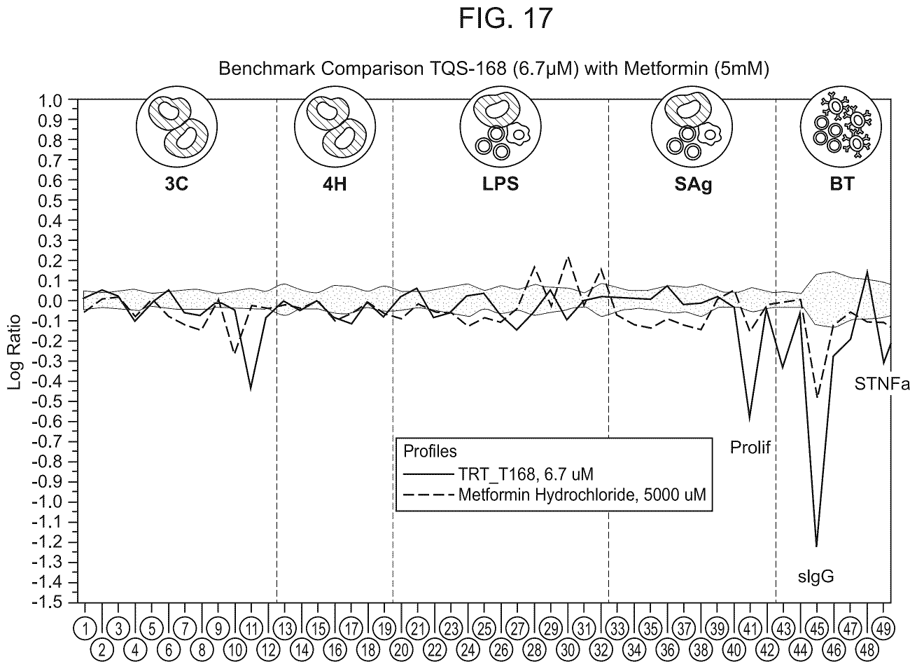

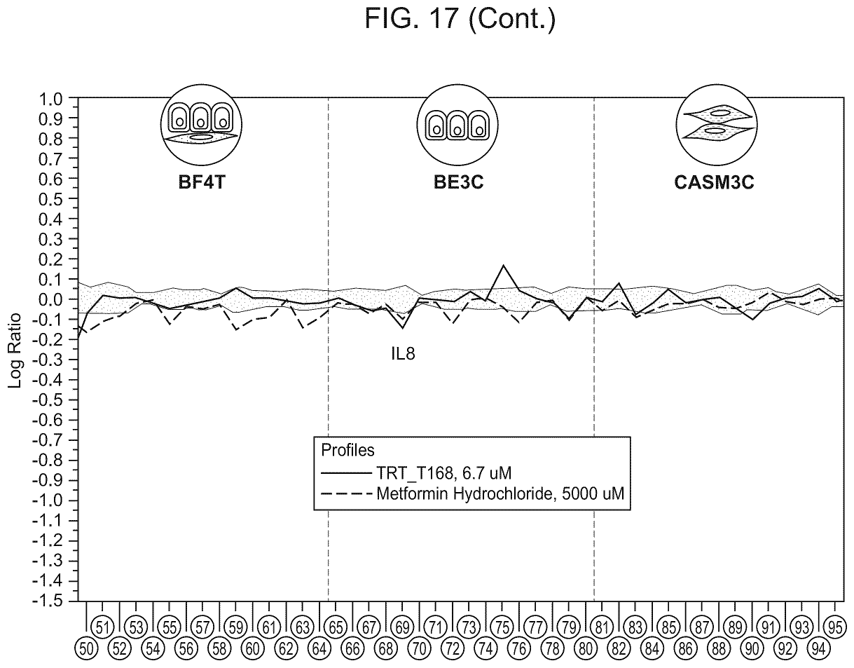

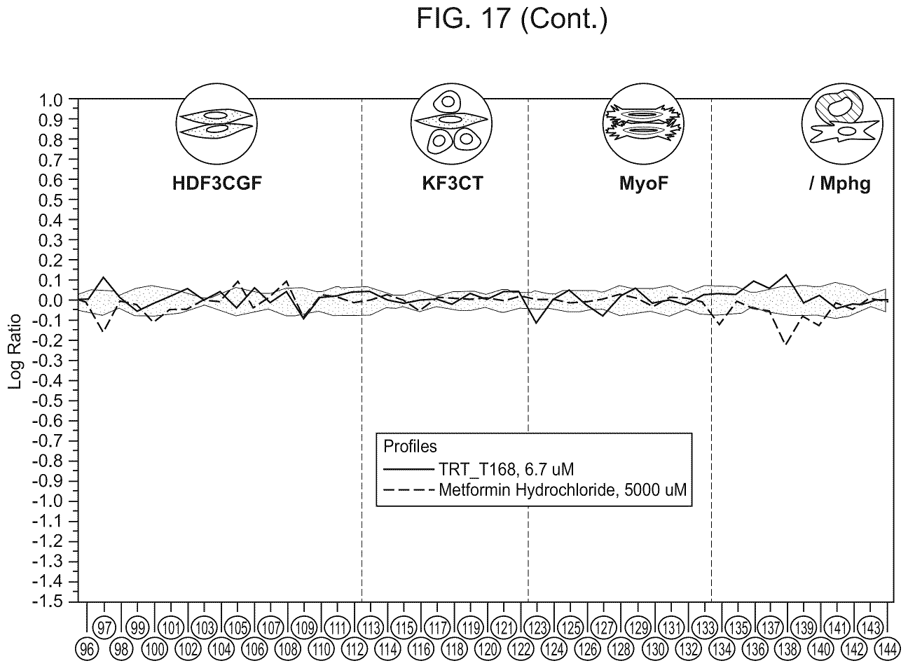

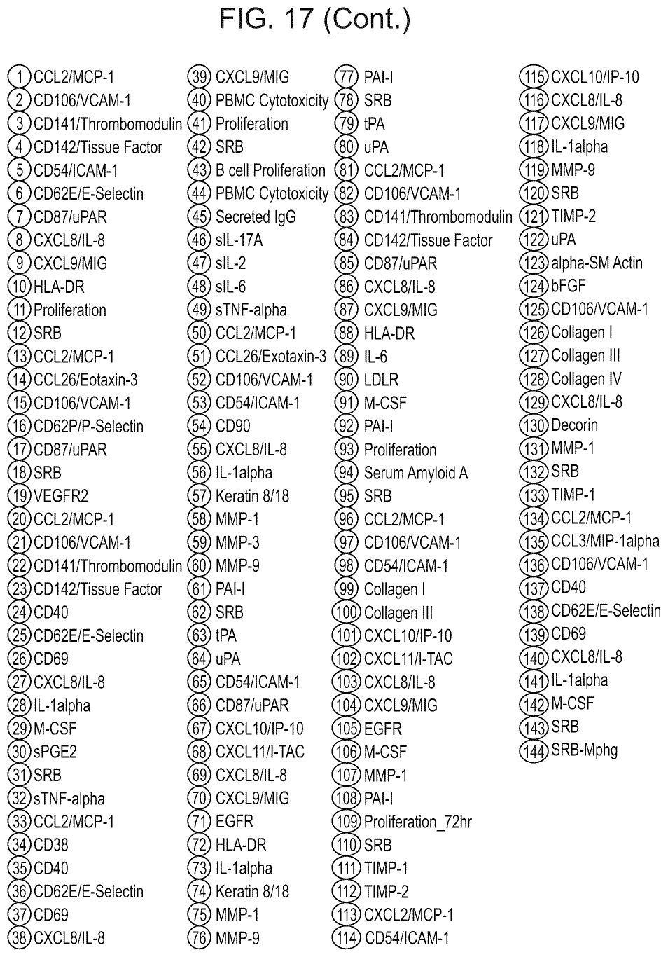

A BioMAP profile was also generated for TQS-168 at a concentration of 6.7 μM and compared with metformin hydrochloride at a concentration of 5000 μM (FIG. 17). Common biomarker readouts were annotated when the readout for both profiles was outside of the significance envelope with an effect size >20% (|log₁₀ ratio|>0.1) in the same direction.

The results showed that TQS-168 and metformin both inhibit T-cell proliferation, IgG, TNFα, and IL-8. However, TQS-168 at a low concentration of 6.7 μM showed a greater decrease in sIgG and sTNFα in B and T cells as compared to metformin hydrochloride at a high concentration of 5000 μM. Thus, TQS-168 was distinguishable from metformin. Metformin hydrochloride is an AMPK activator and antihyperglycemic agent that is used as the first-line therapy for the treatment of type 2 diabetes. Metformin is also an activator of PGC1α.

There were four common activities that were annotated within the following systems: Sag (Prolif), BT (sIgG, sTNFα), and BE3C (IL-8). There are 29 differentiating activities (not shown) within the following systems: 3C (Prolif), 4H (uPAR), LPS (CD40, CD69, IL-8, IL-1α, sPGE2, sTNFα), Sag (CD38, CD40, CD69, IL-8), BT (Prolif, sIL-17A, sIL-2, sIL-6), BF4T (Eotaxin 3, IL-8, MMP-3, tPA), BE3C (HLA-DR, MMP-1, MMP-9), HDF3CGF (VCAM-1, Collagen III), MyoF (αSMA), and lMphg (MCP-1, E-selectin, IL-8). Differentiating biomarkers were defined when one profile had a readout outside of the significance envelope with an effect size >20% (|log₁₀ ratio|>0.1), and the readout for the other profile was either inside the envelope or in the opposite direction.

The activities of TQS-168 in the BioMAP systems are indicative of immunomodulatory, anti-fibrotic, and anti-coagulability potential.

### 3.1.9. Example 9—Pilot Study to Evaluate the Therapeutic Effect of TQS-168 and TQS-168 Analogs for the Treatment of Sepsis

LPS, an outer membrane component of Gram negative bacteria, is a potent activator of monocytes and macrophages. LPS is known to be a toll-like-receptor (TLR) 4 agonist. Mice treated with viral TLR agonists followed by LPS exhibit a hyperinflammatory response to LPS that recapitulates many of the aspects of secondary hemophagocytic lymphohistiocytosis (sHLH) (Wang et al., (2019) PNAS 116: (6), pgs 2200-2209, “Wang”). sHLH is a highly mortal complication associated with sepsis.

An animal model as described in Wang is used to test the safety and efficacy of TQS-168 and TQS-168 analogs for the treatment of sepsis and sepsis-related conditions associated with hyperinflammation.

Briefly, mice are anesthetized with a ketamine/xylazine mixture. A virus, such as a coronavirus, influenza virus, rhinovirus, respiratory syncytial virus, metapneumovirus, adenovirus, or a boca virus, is administered. The virus may be administered intranasally dropwise.

Next, LPS diluted in a buffer, such as PBS, is injected intraperitoneally at specific doses.

To provide comparators for assessing the effects of TQS-168 and its analogs, groups of positive control mice are treated with one or more antibodies against pro-inflammatory cytokines of interest: anti-NK1.1 antibody (PK136; Bio X Cell) diluted in PBS is injected at 100 μg per injection (100 μL) retro-orbitally concurrently with each challenge; anti-IFNγ antibody (XMG1.2, Bio X Cell) is diluted in PBS and injected at 500 μg per injection (100 μL) retro-orbitally concurrently with each challenge; anti-IL6R (15A7.10 mg/kg; Bio X Cell), anti-TNFα (XT3.11, 500 μg per injection; Bio X Cell), anti-IFNαR (MAR1-5A3, 10 mg/kg; Bio X Cell), and anti-IL1β (B122, 500 μg per injection; Bio X Cell) are injected retro-orbitally. For a negative control, isotype antibody is injected retro-orbitally.

Mice in the experimental groups are treated with various doses of TQS-168 or TQS-168 analog to test the efficacy of TQS-168 and TQS-168 analogs in the treatment of inflammation associated with sepsis. The TQS-168 or TQS-168 analogs are administered to the animal through an intravenous route of administration. Various concentrations of TQS-168 and TQS-168 analogs are tested, followed by measurement of cytotoxic effects at the different concentrations.

Plasma cytokines, biomarkers, and complete blood count are analyzed following administration of virus, LPS, antibodies, and TQS-168 or TQS-168 analogs. The pro-inflammatory cytokines can be measured using the ELISA assay described in Example 1.

Primary endpoints include, but are not limited to, immunomodulatory activities following treatment with TQS-168 and TQS-168 analogs at non-cytotoxic concentrations, such as inhibition of pro-inflammatory cytokines TNFα, INFγ, IL-6, IL-10, IL-12 MCP-1, IL-17; toxicity, permeability, solubility, stability, and pharmacokinetic properties; and reduction in in vivo symptoms.

Results: TQS-168 and analogs TQS-235, TQS-237, and TQS-240 are effective in reducing hyperinflammatory symptoms and circulating concentrations of pro-inflammatory cytokines, predicting efficacy in treating sepsis and other systemic inflammatory activation syndromes, including CRS.

### 3.1.10. Example 10—Metabolite TQS-621 Inhibits LPS-Stimulated Release of Pro-Inflammatory Cytokines from Human PBMCs

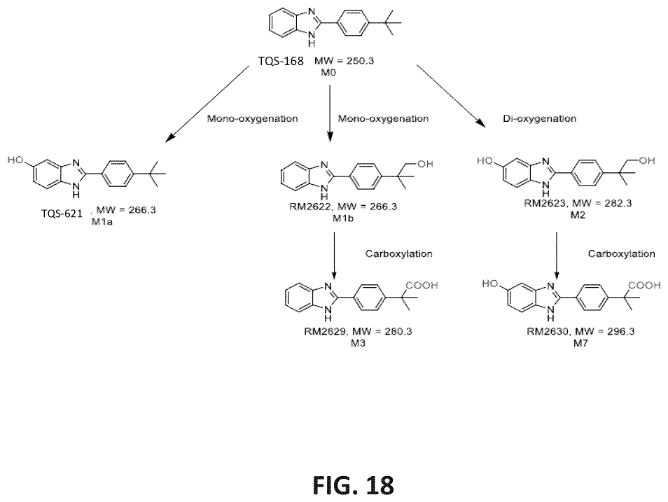

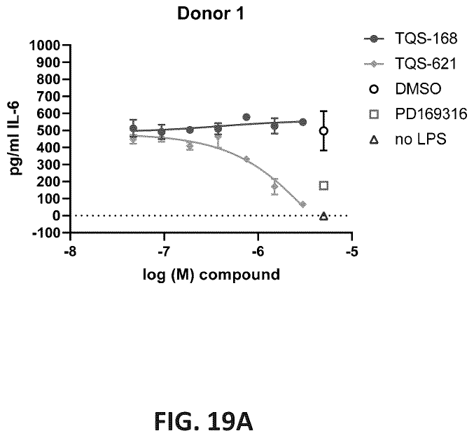

The principal metabolites from liver metabolism of TQS-168 following oral administration have been described. See Sun et al., Rapid Commun. Mass Spectrum. 32:480-488 (2018), incorporated herein by reference. We synthesized the phase 1 metabolites from liver metabolism of TQS-168 illustrated in FIG. 18 and tested them for the ability to inhibit LPS-stimulated secretion of pro-inflammatory cytokines in vitro.

Briefly, 20,000 human PBMC were aliquoted into each well. LPS was added at 1 ng/ml and the cells incubated for 24h in the presence of LPS and TQS-168 or one of its metabolites. Readouts were human IL-6 ELISA and human TNFα ELISA. For assay, supernatants were diluted in culture medium. The supernatant dilution for the IL-6 ELISA was 1:8, and 1:2 for the human TNFα ELISA.

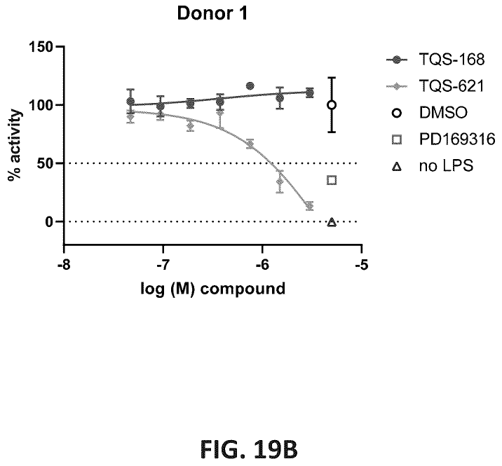

IL-6 results are shown in FIGS. 19A and 19B. FIG. 19A shows absolute inhibition by TQS-168 and metabolite TQS-621 of LPS-stimulated IL-6 secretion from PBMCs obtained from a first donor. The structure of TQS-621 is shown below as Formula II:

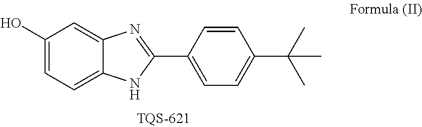 [SMILES: CC(C)(C)c1ccc(-c2nc3cc(O)ccc3[nH]2)cc1]

FIG. 19B shows the relative inhibition of IL-6 inhibition expressed as percentage activity.

### 3.1.11. Example 11—TQS-168 and Metabolite TQS-621 Inhibit Cytokine Release of PBMCs from an ALS Patient

PBMCs from an ALS patient were used for this study. Whole blood samples were collected from the patient and processed for PBMC isolation with Ficoll gradient method. PBMC samples were stored in liquid nitrogen at −80° C. for subsequent analysis of IL-6 production.

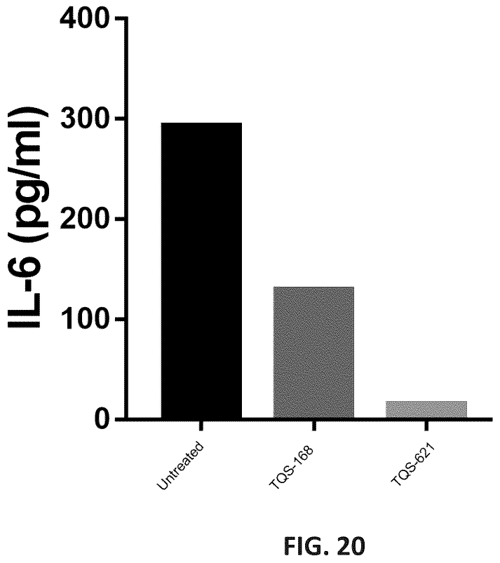

Frozen PBMC samples were thawed and rested at 37° C. PBMCs from ALS patients are known to exhibit spontaneous cytokine release of IL-6 and TNFα. Therefore PBMC stimulation by LPS was not required to evaluate the effects of TQS-168 and metabolite TQS-621 on IL-6 production. PBMC samples (5×10⁵ cells per well) were incubated with medium only, TQS-168 (1 μM) or TQS-621 (1 μM). After 24 hours, supernatants were collected for quantification of IL-6 levels by ELISA. Results are shown in FIG. 20

FIG. 20 shows inhibition of spontaneous release of IL-6 by both TQS-168 and metabolite TQS-621. PBMCs incubated in medium only spontaneously produced IL-6, while incubation with TQS-168 or TQS-621 elicited a reduction in amount of IL-6 produced.

### 4. Equivalents

While various specific embodiments have been illustrated and described, the above specification is not restrictive. It will be appreciated that various changes can be made without departing from the spirit and scope of the invention(s). Many variations will become apparent to those skilled in the art upon review of this specification.

### 5. Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

1. A method of treating a patient who has, or who is at risk for developing, systemic immune activation, comprising:
administering to the patient an effective amount of a compound of Formula I: 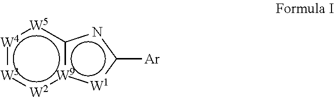
or a salt, hydrate, deuterated analog, or fluorinated analog thereof,
wherein:
Ar is 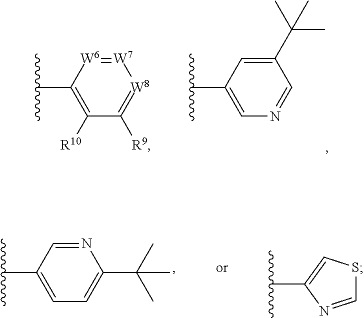
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 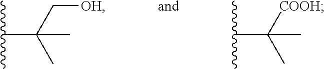
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 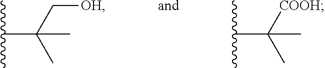
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxoalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.

2. The method of claim 1, wherein systemic immune activation does not comprise clinically meaningful neuroinflammation.

3. The method of claim 1, wherein the compound of Formula I is selected from: 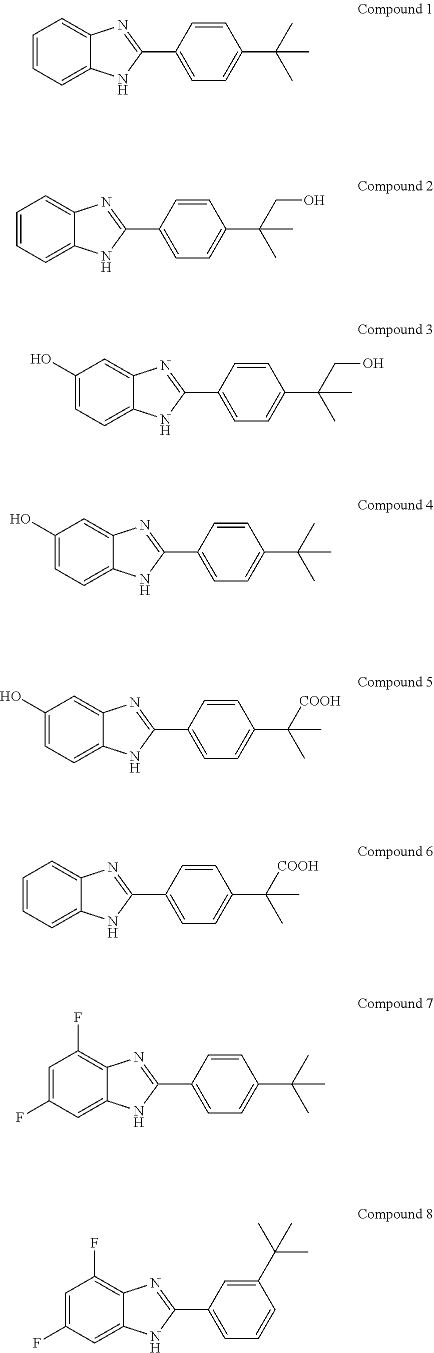 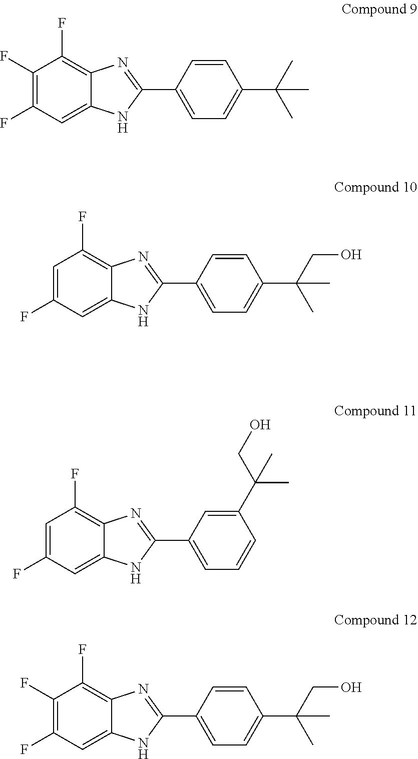 [SMILES: CC(C)(C)c1ccc(-c2nc3c(F)c(F)c(F)cc3[nH]2)cc1 ; CC(C)(CO)c1ccc(-c2nc3c(F)cc(F)cc3[nH]2)cc1 ; CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]
or a salt, hydrate, deuterated analog, or fluorinated analog thereof.

4-8. (canceled)

9. The method of claim 1, wherein the patient has cytokine release syndrome (CRS), acute lung inflammation (ALI), acute respiratory distress syndrome (ARDS), ALI with concomitant pneumonia, or ARDS with concomitant pneumonia.

10-12. (canceled)

13. The method of claim 1, wherein the patient has acute renal injury.

14-15. (canceled)

16. The method of claim 1, wherein the patient has sepsis and administering the compound of Formula I reduces symptomatic immune activation.

17-26. (canceled)

27. The method of claim 1, wherein the compound of Formula I is administered intravenously or enterically.

28. The method of claim 27, wherein the compound of Formula I is administered by mouth (p.o.).

29-31. (canceled)

32. The method of claim 1, wherein the effective amount of the compound of Formula I is between 0.5 mg/kg and 85 mg/kg per day by mouth.

33. (canceled)

34. The method of claim 32, wherein the dose is administered as a single daily dose.

35-36. (canceled)

37. The method of claim 1, wherein the patient has a body temperature greater than 37.5° C. prior to first administration of the compound of Formula I, or a salt, hydrate, deuterated analog, or fluorinated analog thereof.

38. (canceled)

39. The method of claim 1, wherein the patient has a pre-treatment C-reactive protein (CRP) level greater than 2 mg/L.

40. (canceled)

41. The method of claim 1, wherein the patient has a pre-treatment serum IL-6 level of at least 2 pg/ml.

42-49. (canceled)

50. The method of claim 1, wherein the method reduces the patient's serum CRP levels below pre-treatment levels.

51-52. (canceled)

53. The method of claim 1, wherein the method reduces, in the patient, one or more pro-inflammatory cytokine serum levels below pre-treatment levels, wherein the one or more pro-inflammatory cytokines is selected from the group consisting of: IL-6, TNFα; IL-17A; IL-17F, and IL-2.

54. The method of claim 53, wherein the method reduces the patient's serum IL-6 levels below pre-treatment levels.

55. The method of claim 54, wherein the serum IL-6 level is decreased by at least 10% as compared to pre-treatment levels.

56. (canceled)

57. The method of claim 53, wherein the serum TNFα level is decreased by at least 10% as compared to pre-treatment levels.

58. The method of claim 53, wherein the serum TNFα level is decreased by at least 20% as compared to pre-treatment levels.

59-69. (canceled)

70. The method of claim 1, wherein the administration of the compound of Formula I, or a salt, hydrate, deuterated analog, or fluorinated analog thereof increases the expression level of PCG-1α in the lungs as compared to pre-treatment levels.

71-132. (canceled)

133. A method of treating a patient who has, or who is at risk for developing, sepsis, comprising:
administering to the patient an effective amount of a compound of Formula I: 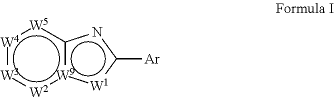
or a salt, hydrate, deuterated analog, or fluorinated analog thereof,
wherein:
Ar is 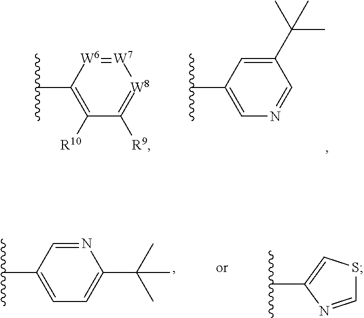
W¹ is chosen from N—R¹, O, and S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is selected from H, (C₁-C₃)alkyl, —CH₂OC(═O)R³⁰, —CH₂OP(═O)OR⁴⁰OR⁴¹, —C(═O)OR⁴², and —C(═O)R⁴³;
R², R³, R⁴, and R⁵ are selected independently from hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁-C₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, and (C₁-C₄)acylamino;
R⁶ and R¹⁰ are selected independently from hydrogen, deuterium, halo, (C₁-C₃)alkyl, perfluoro(C₁-C₃)alkyl, hydroxy, (C₁-C₃)alkoxy, perfluoro(C₁-C₃)alkoxy, and amino;
R⁷ and R⁹ are selected independently from hydrogen, deuterium, hydroxy, cyano, amino, halogen, halo(C₁-C₄)alkyl, (C₁-C₄)alkoxy, halo(C₁-C₄)alkoxy, 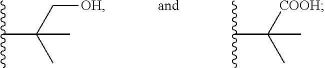
R⁸ is selected from hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 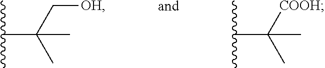
R³⁰ is selected from (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
R⁴⁰ and R⁴¹ are selected independently from hydrogen (C₁-C₆)hydrocarbyl;
R⁴² is (C₁-C₅)alkyl;
R⁴³ is (C₁-C₃)alkyl,
R⁴⁴ is selected from any naturally occurring amino acid sidechain;
R⁴⁵ is selected from H, methyl, and (C₁-C₄)alkoxycarbonyl; and
R⁵⁰ is H or (C₁-C₃)alkyl.


SEQUENCE LISTING (1 sequence)

SEQ ID NO: 1 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP